SEQUENCE LISTING (6 sequences)

SEQ ID NO: 1 - DNA, 408 bp, Mus musculus
tcttgcccatcggggccacggatacctgtgtgtcctccccgccattccatgcccaacggggttttggatacttacctgccttttcattctttttttttcttattatttttttttctaaacttgcccatctgggctgtggatacctgcttttattctttttttcttctccttagcccatcggggccatggatacctgctttttgtaaaaaaaaaaaaaaaaacaaaaaaacctttctcggtccatcgggacctcggatacctgcgtttagtctttttttcccatgcccaacggggcctcggatacctgctgttattatttttttttctttttcttttgcccatcggggctgtggatacctgctttaaattttttttttcacggcccaacggggcgcttggtggatggaa

SEQ ID NO: 2 - DNA, 395 bp, R. norvegicus
ttttgcccatcggggccacggatacctgtgtgtcctcccagccattccatgtccagctgggcttgggatacttaacctgccttttaatccttttttcttcttcttacttttcttcttctaaacttgcccatctgggttgtggatacctgcttttattctttttttcttcttctccttagcccatcggggccatggatacctgctttttaccaaaaaacgccgtatttctcggtccatcgggacctcggatacctgcgtttagtttttttcccatgcccaacggggcctcggatacctgctttaatttttttttcttttccttttgcccatcggggctgtggatacctgctttaattttttttttcacggcccatcggggcatttggtggatggaa

SEQ ID NO: 3 - DNA, 434 bp, Felis catus
ttttgcccatcggggctgtggataccaggttttattatcatttttttcgcccaacggggctgtggatacctgcgttttaattcttttcttttatttattttttttaatttgcccatcggggcagcggatacctgcttttaattttttttttttttcacccttagcccatcggggcctcggatacctgctgtgtctccttccctccccttaaccctctggcccatcggggcaatggataccagctttaaaaaaagttcctttttggcccatcggggcctcggatacctgcttttattattttttttcccttgcccatcggggccttggatacctgctttatttatttattttttttcctttgcccatcggggctgtggatacctgcttagatttttttttttctcatggcccatcggggccttttatggatggaa

SEQ ID NO: 4 - DNA, 408 bp, Homo sapiens
ttttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaa

SEQ ID NO: 5 - RNA, 15 nt, Artificial sequence
  Synthetic oligonucleotide
  misc_feature (1)..(15): y is c or t
  misc_feature (1)..(15): n is any base
nngyccaucggggyn

SEQ ID NO: 6 - RNA, 13 nt, Artificial sequence
  Synthetic oligonucleotide
  misc_feature (1)..(13): n is any base
  misc_feature (1)..(13): y is c or t
nyggauaccugnn

(19) United States
(12) Patent Application Publication
(10) Pub. No.: US 2022/0408702 A1
(43) Pub. Date: Dec. 29, 2022

(54) DIRECTED CHANGES IN ORGANISM POPULATIONS

(71) Applicants: Massachusetts Institute of Technology, Cambridge, CA (US); Trustees of Tufts College, Medford, MA (US)

(72) Inventors: Kevin Michael Esvelt, Cambridge, MA (US); Sam Rountree Telford, III, Shrewsbury, MA (US)

(73) Assignees: Massachusetts Institute of Technology, Cambridge, MA (US); Trustees of Tufts College, Medford, MA (US)

(21) Appl. No.: 17/778,482

(22) PCT Filed: Nov. 20, 2020
(86) PCT No.: PCT/US20/61504

§ 371 Date: May 20, 2022

Related U.S. Application Data
(60) Provisional application No. 62/938,532, filed on Nov. 21, 2019.

(51) Int. Cl. A01K 67/027; C12N 15/10

(52) U.S. Cl. CPC A01K 67/0275; C12N 15/102; A01K 2217/07; A01K 2217/30; A01K 2227/105; A01K 2267/02

(57) ABSTRACT

The invention relates, in part, to methods to introduce gene alleles of interest into members of a population of a species. The invention also relates to methods to prepare large numbers of organisms comprising one or more introduced alleles of interest in an otherwise wild-type genome.

### RELATED APPLICATIONS

This application claims benefit under 35 U.S.C § 119(e) of U.S. Provisional application Ser. No. 62/938,532 filed Nov. 21, 2019, the disclosure of which is incorporated by reference herein in its entirety.

### SEQUENCE LISTING

The instant application incorporates by reference the Sequence Listing in the ASCII text file filed Nov. 21, 2020, entitled “MIT-028WO(01)_ST25.txt”, which file was created on Nov. 18, 2020 the size of which file is 3,513 bytes.

### FIELD OF THE INVENTION

The invention relates, in part, to methods for introducing engineered organisms exhibiting preselected heritable phenotypic and/or preselected heritable genotypic traits into organism populations.

### BACKGROUND OF THE INVENTION

There are currently no available methods for releasing engineered organisms other than insects into the wild. For insects, only sterile or autosomal female-lethal releases are used. In the U.S. the only approved examples are Oxitec sterile male mosquitoes and female-lethal diamondback moths, as well as MosquitoMate/Verily Wolbachia-infected sterile male mosquitoes. A drawback of current insect-based strategies is their reliance on mass rearing techniques that typically impair fitness in the wild due to inbreeding and subsequent loss of genetic diversity, genetic adaptation to captivity, or loss of behavioral adaptations that are only transmitted in the wild. In addition, current methods can only release organisms that do not directly cause harm (e.g. sterile insects where only the larvae cause harm, or male mosquitoes that do not bite). Current methods do not provide effective or efficient options for use in non-insect organisms.

### SUMMARY OF THE INVENTION

According to an aspect of the invention a method of altering or changing a genetic characteristic of a target population of organisms of a species is provided, the method including: (a) preselecting a heritable trait for introduction into a target population of organisms; and (b) introducing by birth into the target population an engineered organism that includes the preselected heritable trait, wherein the introduction changes a genetic characteristic of the target population. In some embodiments, a means of introducing the preselected heritable trait by birth includes: (i) delivering into a reproductive cell of a captive female of the organism a gene or gene allele that when expressed in an descendant of the captive female organism results in an engineered organism that exhibits the preselected heritable trait; (ii) impregnating the captive female of the organism; and (iii) releasing the impregnated captive female of the organism into the target population of organisms. In some embodiments, the means of the delivery in (a) impregnates the captive female. In certain embodiments, the captive female organism is obtained from the target population of organisms. In certain embodiments, the captive female organism is not obtained from the target population of organisms. In some embodiments, the captive female is obtained from a wild population of the species. In some embodiments, the captive female organism is a captivity-raised female organism. In some embodiments, the engineered organism is a male engineered organism. In some embodiments, the engineered organism is born in a wild population of organisms of the species. In certain embodiments, the engineered organism is born in captivity. In some embodiments, the target population is a captive population of organisms of the species. In certain embodiments, the target population is a wild population of organisms of the species. In some embodiments, the preselected heritable trait is detected in an engineered organism descendant of the impregnated captive female organism released into the target population. In some embodiments, the preselected heritable trait is a preselected heritable phenotypic trait. In certain embodiments, the organism is a mammal. In some embodiments, the organism is of the genus Rattus, Mus, Sus, Felis or Canis. In some embodiments, the organism is of the order Rodentia. In some embodiments, the organism is a rat. In certain embodiments, the organism is a mouse, and optionally is a mouse of the genus Mus or the genus Peromyscus or the genus Mastomys. In some embodiments, the mouse is a white-footed mouse (P. leucopus). In some embodiments, the preselected heritable trait includes a coat color of the organism, and results from expression of a coat-color allele. In certain embodiments, the coat-color allele includes a visible dominant (blaze) allele. In some embodiments, the coat-color allele includes a visible recessive (Oca2^{P}, Tyr^{c-ch}) allele. In certain embodiments, the preselected heritable trait is a daughterless-male heritable trait. In some embodiments, the daughterless-male heritable trait is generated by engineering a Y chromosome that when present in a female of the organism disrupts an activity of an X chromosome non-coding RNA gene. In some embodiments, the daughterless-male heritable trait includes disruption of an activity of an X chromosome non-coding RNA gene in female descendants of an engineered male organism, wherein the disruption of the activity of the X chromosome non-coding RNA gene in the female descendants is embryonically lethal to the female descendants. In some embodiments, a means of producing the descendant organisms including the heritable trait comprises impregnating a female organism of the species with genetic material of the engineered male. In certain embodiments, the method also includes releasing the engineered male organism into a target population of organisms of the species. In some embodiments, a means for the disrupting of an activity of the X chromosome non-coding RNA gene in female descendants of the engineered male organism includes encoding on the engineered male organism's Y chromosome a nuclease capable of disrupting the activity of the X chromosome non-coding RNA gene. In certain embodiments, the nuclease is an RNA-guided nuclease. In some embodiments, one or more guide RNAs are encoded within one or more introns of the encoded nuclease. In certain embodiments, the nuclease is capable of cutting on both sides of one or more stem-loop repeats in the X chromosome non-coding RNA gene. In some embodiments, the nuclease is capable of cutting within a stretch of stem-loop repeats. In some embodiments, the nuclease is encoded in a translational fusion to an endogenous gene in the engineered male organism. In certain embodiments, the endogenous gene is expressed in the germline of males of the organism. In some embodiments, the endogenous gene is a Y-chromosomal gene. In some embodiments, the endogenous gene is Eif2s3y, Zfy, or Ddx3y. In certain embodiments, the method also includes inserting a plurality of introns into the coding region of the endogenous gene, wherein the inserted plurality of introns increases a level of translation of the endogenous gene compared a level of translation of the endogenous gene in the absence of the inserted plurality of introns. In certain embodiments, the translational fusion includes an N-terminal 2A-peptide fusion to the endogenous gene. In some embodiments, the N-terminal 2A-fusion to the endogenous gene comprises a modified Kozak sequence, wherein the modified Kozak sequence increases a level of translation of the endogenous gene compared to a level of translation of the endogenous gene in the absence of the modified Kozak sequence. In some embodiments, the method also includes one or both of: (a) inserting a plurality of introns into the coding region of the endogenous gene and (b) inserting a modified Kozak sequence into the N-terminal 2A fusion protein, wherein one or both of (a) and (b) increases a level of translation of the endogenous gene compared a level of translation of the endogenous gene in the absence of (a), (b), or both (a) and (b), respectively. In certain embodiments, one or more introns in the plurality introns is a synthetic intron. In some embodiments, one or more of the introns in the plurality of introns is native to a gene of the organisms of the species and is included in a sequence encoding the nuclease. In some embodiments, the method also includes, encoding one or more independently selected polymerase III promoters upstream of the one or more encoded guide RNAs, wherein the polymerase III promoter(s) enhances the expression of the one or more guide RNAs compared to the expression of the one or more guide RNAs in the absence of the encoded polymerase III promoter(s). In some embodiments, the polymerase III promoter is a U6 promoter, a 7SK promoter, an H1 promoter, or a tRNA promoter. In some embodiments, the one or more encoded polymerase III promoters are independently selected. In some embodiments there are two, three, four, five, six, or more independently selected encoded polymerase III promoters, of which two or more are the same as each other, or of which each is different from the others. In certain embodiments, the polymerase III promoter is a synthetic promoter. In some embodiments, the polymerase III promoter is a polymerase III promoter native to the organism species. In some embodiments, the polymerase III promoter is a polymerase III promoter not native to the organism species. In some embodiments, the X chromosome non-coding RNA gene is an X-inactive specific transcript (Xist) gene. In certain embodiments, the disruption of the Xist gene includes deletion of at least a portion of the Xist gene. In some embodiments, the disruption of the Xist gene includes deletion of one, two, three, or more stem-loop repeats in Xist region A within exon 1 of the Xist gene. In certain embodiments, the preselected heritable trait is a sterile-daughter heritable trait. In some embodiments, the sterile-daughter heritable trait is generated by engineering a Y chromosome that disrupts an activity of an X chromosome gene required for the fertility of female offspring. In some embodiments, the sterile-daughter heritable trait is generated by engineering a Y chromosome that produces a nuclease carried in sperm of the engineered male organism and when present in a zygote, the nuclease cuts one or more female-specific fertility genes. In certain embodiments, the method also includes preparing a zygote that includes the nuclease. In some embodiments, a means of preparing the zygote that includes the nuclease includes impregnating a female of the organism with genetic material of a sperm of the engineered male organism. In some embodiments, the impregnated female organism includes one or more of the zygotes that include the nuclease. In certain embodiments, the female-specific fertility genes include Afp, Il11ra1, Dlgap5, Juno, Kpna6, Cdc25b, Hsd17b1, FIGLA, Kpna7, Dppa3, NPM2, NOBOX, GDF9, OOEP, ZP3, ZAR1, Bmp15, NLRP5, PAD6, Filia, TLE6, PgR, Prl, Tnfaip6, Gja4, FshR, Fsh, Aaas, Adgrd1, Ambp, Antxr2, Camk2g, Cdc25b, Cpa2, Creld2, Fmn2, Igfbp7, Kpna2, Mir200b, Mir429, Pappa, Ptx3, Wnt6, Akr1c18, Cuzd1, Ddr1, Acls4, Pcytb1, Sat1, Sox17, or Tkt1. In some embodiments, the sterile-daughter trait includes disruption of an activity of an X chromosome gene in female descendants of an engineered male organism, wherein the disruption of the activity of the X chromosome gene in the female descendants results in sterile female descendants. In some embodiments, the delivering of the gene or gene allele into the reproductive cell of the captive female organism includes an in vitro fertilization (IVF) method. In certain embodiments, the delivering of the gene or gene allele into the reproductive cell of the captive female organism includes an artificial insemination method. In some embodiments, the delivering of the gene or gene allele into the reproductive cell of the captive female organism includes mating of the captive female organism with a male organism of the species. In certain embodiments, the method also includes detecting the preselected heritable trait in an organism in the target population following the introduction by birth of the preselected heritable trait into the target population. In some embodiments, the preselected heritable trait includes a genotypic trait and a method of detecting the preselected heritable trait comprises a sequencing method. In some embodiments, the sequencing includes sequencing DNA obtained from an organism of the species obtained from the target population following the introduction by birth of the preselected heritable trait into the target population. In certain embodiments, the preselected heritable trait includes a phenotypic trait and a method of detecting the preselected heritable trait includes a visual detection means. In some embodiments, a method of detecting the preselected heritable trait includes detecting a genotypic trait in an organism of the species following the introduction by birth of the preselected heritable trait into the target population, wherein the presence of the trait indicates the presence of the genotypic trait in the obtained organism. In some embodiments, the detecting occurs at least 1, 2, 3, 4, 5, 6, 7, 8, 9, 10, 11, 12, 13, 14, 15, 16, 17, 18, 19, 20, 40, 60, 80, 100, or 200 weeks after the release of the impregnated captive female organism. In certain embodiments, the method also includes repeating the detecting at least 1, 2, 3, 4, 5, 6, 7, 8, 9, or 10 additional times. In some embodiments, the method also includes repeating the steps (i)-(iii) in 1, 2, 3, 4, 5, 6, 7, 8, 9, 10, or more captive females of the organism. In some embodiments, the method also includes introducing by birth 1, 2, 3, 4, 5, 6, 7, 8, 9, 10, or more of additional preselected heritable trait(s) into the target population. In certain embodiments, the additional preselected heritable trait(s) are independently selected. In some embodiments, the method also includes capturing one or more female organisms from the target population, at a time subsequent to the release of the impregnated captive female, wherein the capturing is aided in part by a determined spatial distribution of engineered organisms introduced by birth into the target population. In some embodiments, the engineered organism comprises a gene-drive system. In certain embodiments, the organism is not a human.

According to another aspect of the invention, a method of altering a genetic characteristic of a target population of organisms of a species is provided the method including: (a) obtaining one or a plurality of captive female organisms of a species, wherein the species is a non-human species; (b) introducing a genetic element from one or more male organisms of the species into the one or the plurality of the captive female organisms, wherein each of the one or more male organisms exhibits one or more preselected heritable traits; wherein the introducing results in impregnation of at least one of the captive female organisms, and a descendant of the impregnated captive female organism exhibits one or more of the preselected heritable traits; (c) identifying one or more of the captive female organisms impregnated in (b); and (d) releasing one or more of the identified impregnated captive female organisms into a target population of organisms of the species, wherein the presence of one or more descendants of the impregnated captive female organisms alters a genetic characteristic of the target population. In certain embodiments, the preselected heritable trait includes one or more of a preselected heritable phenotypic trait and a preselected heritable genotypic trait. In some embodiments, at least one of the preselected heritable phenotypic and the heritable genotypic trait is detected in an engineered descendant of the impregnated captive female organism released into the target population. In certain embodiments, the method also includes preselecting the one or more heritable traits, wherein the preselected heritable traits are of interest to introduce into the target population of organisms of the species. In some embodiments, the organism is a mammal. In some embodiments, the organism is of the genus Rattus, Mus, Sus, Felis or Canis. In certain embodiments, the organism is of the order Rodentia. In some embodiments, the organism is a rodent. In some embodiments, the organism is a rat. In certain embodiments, the organism is a mouse, and optionally is a mouse of the genus Mus or the genus Peromyscus or the genus Mastomys. In some embodiments, the mouse is a white-footed mouse (P. leucopus). In some embodiments, the preselected heritable trait includes a coat color. In certain embodiments, the preselected heritable trait includes a coat-color allele. In some embodiments, the coat-color allele includes a visible dominant (blaze) allele. In some embodiments, the coat-color allele includes a visible recessive (Oca2^{P}, Tyr^{c-ch}) allele. In certain embodiments, the genetic element includes a gene or gene allele. In certain embodiments, the introduction of the genetic element from one or more male organisms of the species into one or a plurality of the captive female organisms includes an in vitro fertilization (IVF) method. In some embodiments, the introduction of the genetic element from one or more male organisms of the species into one or a plurality of the captive female organisms includes an artificial insemination method. In some embodiments, the introduction of the genetic element from one or more male organisms of the species into one or a plurality of the captive female organisms includes mating of the captive female organism with the male organism of the species. In some embodiments, the method also includes detecting one or more of the preselected heritable trait in an organism obtained from the target population following the release of the impregnated captive female organisms into the target population. In certain embodiments, a method of detecting the preselected heritable trait comprises a sequencing method. In certain embodiments, the sequencing includes sequencing DNA obtained from an organism of the species obtained from the target population following the release of the impregnated captive female organisms into the target population. In some embodiments, a method of detecting the preselected heritable trait includes a visual detection means. In some embodiments, a method of detecting the preselected heritable trait includes detecting a genotypic trait in an organism of the species obtained from the target population following the release of the impregnated captive female organisms into the target population, wherein the presence of the trait indicates the presence of the genotypic trait in the obtained organism. In certain embodiments, the detecting occurs at least 1, 2, 3, 4, 5, 6, 7, 8, 9, 10, 11, 12, 13, 14, 15, 16, 17, 18, 19, 20, 40, 60, 80, 100, or 200 weeks after the release of the impregnated captive female organisms. In some embodiments, the method also includes repeating the detecting at least 1, 2, 3, 4, 5, 6, 7, 8, 9, or 10 additional times. In some embodiments, the captive female organism is a wild-caught female organism of the species. In certain embodiments, the captive female organism is a captivity-raised female organism. In some embodiments, the target population is a wild population of organisms of the species. In some embodiments, the target population includes a population of organisms of the species from which the captive female organism was obtained. In certain embodiments, the target population is not a population of organisms of the species from which the captive female organism was obtained. In some embodiments, the target population is a captive population of the species. In some embodiments, altering the genetic characteristic of the target population includes the exhibition of the one or more of the preselected heritable traits in one or more organisms in the target population. In some embodiments, the method also includes capturing one or more female organisms from the target population, at a time subsequent to the release of the impregnated captive female, wherein the capture is aided in part by a determined spatial distribution of one or more the engineered organisms introduced into the target population. In certain embodiments, the one or more engineered organisms are introduced by birth into the target population. In some embodiments, the preselected heritable trait is a daughterless-male heritable trait. In some embodiments, the daughterless-male heritable trait is generated by engineering a Y chromosome that when present in a female of the organism disrupts an activity of an X chromosome non-coding RNA gene. In certain embodiments, the nuclease is an RNA-guided nuclease. In some embodiments, one or more guide RNAs are encoded within one or more introns of the encoded nuclease. In certain embodiments, the nuclease is capable of cutting on both sides of one or more stem-loop repeats in the X chromosome non-coding RNA gene. In some embodiments, the nuclease is capable of cutting within a stretch of stem-loop repeats. In some embodiments, the nuclease is encoded in a translational fusion to an endogenous gene in the engineered male organism. In certain embodiments, the endogenous gene is expressed in the germline of males of the organism. In some embodiments, the endogenous gene is a Y-chromosomal gene. In some embodiments, the endogenous gene is Eif2s3y, Zfy, or Ddx3y. In some embodiments, the method also includes inserting a plurality of introns into the coding region of the endogenous gene, wherein the inserted plurality of introns increases a level of translation of the endogenous gene compared a level of translation of the endogenous gene in the absence of the inserted plurality of introns. In certain embodiments, the translational fusion includes an N-terminal 2A-peptide fusion to the endogenous gene. In some embodiments, the N-terminal 2A-fusion to the endogenous gene comprises a modified Kozak sequence, wherein the modified Kozak sequence increases a level of translation of the endogenous gene compared to a level of translation of the endogenous gene in the absence of the modified Kozak sequence. In certain embodiments, the method also includes one or both of: (a) inserting a plurality of introns into the coding region of the endogenous gene and (b) inserting a modified Kozak sequence into the N-terminal 2A fusion protein, wherein one or both of (a) and (b) increases a level of translation of the endogenous gene compared a level of translation of the endogenous gene in the absence of (a), (b), or both (a) and (b), respectively. In some embodiments, one or more introns in the plurality introns is a synthetic intron. In some embodiments, one or more of the introns in the plurality of introns is native to a gene of the organisms of the species and is included in a sequence encoding the nuclease. In certain embodiments, the method also includes, encoding one or more polymerase III promoters upstream of the one or more encoded guide RNAs, wherein the one or more polymerase III promoter enhances the expression of the one or more guide RNAs compared to the expression of the one or more guide RNAs in the absence of the encoded polymerase III promoter(s). In some embodiments, the one or more encoded polymerase III promoters are independently selected. In some embodiments, there are two, three, four, five, six, or more independently selected encoded polymerase III promoters, of which two or more are the same as each other, or of which each is different from the others. In some embodiments, the polymerase III promoter is a U6 promoter, a 7SK promoter, an H1 promoter, or a tRNA promoter. In some embodiments, the polymerase III promoter is a synthetic promoter. In some embodiments, the polymerase III promoter is a polymerase III promoter native to the organism species. In certain embodiments, the polymerase III promoter is a polymerase III promoter not native to the organism species. In some embodiments, the X chromosome non-coding RNA gene is an X-inactive specific transcript (Xist) gene. In some embodiments, the disruption of the Xist gene includes deletion of at least a portion of the Xist gene. In certain embodiments, the disruption of the Xist gene includes deletion of one, two, three, or more stem-loop repeats in Xist region A within exon 1 of the Xist gene. In some embodiments, the daughterless-male heritable trait includes disruption of an activity of an X chromosome non-coding RNA gene in female descendants of an engineered male organism, wherein the disruption of the activity of the X chromosome non-coding RNA gene in the female descendants is embryonically lethal to the female descendants. In some embodiments, the X chromosome non-coding RNA gene is an X-inactive specific transcript (Xist) gene. In some embodiments, the disruption of the Xist gene includes deletion of at least a portion of the Xist gene. In certain embodiments, the disruption of the Xist gene comprises deletion of one, two, three, or more stem-loop repeats in Xist region A within exon 1 of the Xist gene. In some embodiments, the preselected heritable trait is a sterile-daughter heritable trait. In certain embodiments, the sterile-daughter heritable trait is generated by engineering a Y chromosome that produces a nuclease carried in the sperm and deposited into the zygote, in which it cuts one or more female-specific fertility genes. In some embodiments, the female-specific fertility genes include Afp, Il11ra1, Dlgap5, Juno, Kpna6, Cdc25b, Hsd17b1, FIGLA, Kpna7, Dppa3, NPM2, NOBOX, GDF9, OOEP, ZP3, ZAR1, Bmp15, NLRP5, PAD6, Filia, TLE6, PgR, Prl, Tnfaip6, Gja4, FshR, Fsh, Aaas, Adgrd1, Ambp, Antxr2, Camk2g, Cdc25b, Cpa2, Creld2, Fmn2, Igfbp7, Kpna2, Mir200b, Mir429, Pappa, Ptx3, Wnt6, Akr1c18, Cuzd1, Ddr1, Acls4, Pcytb1, Sat1, Sox17, and Tkt1. In certain embodiments, the sterile-daughter heritable trait includes disruption of an activity of an X chromosome gene in female descendants of an engineered male organism, wherein the disruption of the activity of the X chromosome gene in the female descendants results in sterile female descendants. In some embodiments, the engineered organism includes a gene-drive system. In some embodiments, the organism is not a human.

### BRIEF DESCRIPTION OF THE DRAWINGS

FIG. 1A-B illustrates sequence features of Xist exon 1. FIG. 1A shows image of sequence alignment of Xist exon 1 region A in mice (M. musculus, SEQ ID NO: 1, shown in all views as top sequence of four), rats (R. norvegicus, SEQ ID NO: 2, shown in all views as second sequence from top of four), cats (F. catus, SEQ ID NO: 3, shown in all views as third sequence from top of four), and humans (H. sapiens, SEQ ID NO: 4, shown in all views as bottom sequence of four). Numbers indicate residue numbers for the sequences in the alignment. The critical stem-loop structural repeats required for functionality are boxed, and correspond to those shown in FIG. 1B. In FIG. 1A, “*” over a box indicates a SEQ ID NO: 5 stem-loop structural repeat, and “#” over a box indicates a SEQ ID NO: 6 stem-loop repeat. Mice and rats harbor 7.5 paired stem-loop repeats, while cats and humans encode 8.5. In mice, 5.5 synthetic repeats are enough for Xist function, while four are insufficient [Wutz, A. et al., (2002) Nature Genetics, 30(2):167-174]. FIG. 1B shows two stem-loop structural repeats, with SEQ ID NO: 5 on left, and SEQ ID NO: 6 on right. These are repeated structural motifs in Xist exon 1 that are required for X-inactivation,

FIG. 2A-B shows schematic diagrams of a Mus Y chromosome encoding a daughterless system and of a screening strategy for sgRNAs used for Xist deletion. FIG. 2A illustrates how a daughterless mouse can be generated by encoding a functional CRISPR system on the Y chromosome such that it disrupts the function of the X-encoded Xist gene in the male germline. FIG. 2B illustrates the screening process for candidate sgRNAs for Xist deletion in Mus cells in which GFP is not expressed unless the 900 base-pair Xist region is deleted.

### DETAILED DESCRIPTION

Aspects of the invention, in part, include methods of introducing one or more of a preselected heritable phenotypic and/or genotypic trait into a population of organisms of a species. Methods of the invention permit efficient introduction of novel alleles conferring new phenotypes into a target population of organisms without the direct addition of organisms with the preselected heritable trait(s) into the population. In certain embodiments of the invention, a female organism is impregnated in a manner that results in offspring of the female that include the preselected heritable phenotypic and/or genotypic trait. The female organism, also referred to herein as a “host” organism may be obtained from a population of interest, impregnated, and released into a target population. In certain methods of the invention, the impregnated female is released into a target population, which may, but need not be, the population from which the female organism was obtained. The release of one or more impregnated female organisms into a target population can result in introduction of the preselected heritable trait(s) into the target population by the birth of genetically engineered offspring in the target population. Thus, certain embodiments of the invention can be used to introduce preselected heritable trait(s) into a target population without adding, except by birth, engineered organisms into the target population.

In some embodiments of the invention, one or more of a preselected heritable phenotypic and/or genotypic trait is introduced into a population of organisms via “engineered” offspring that are born to a host female that has been released into the population. Methods of the invention can be used to introduce one or more preselected heritable traits into a target population of organisms without directly introducing into the target population an existing organism that exhibits the preselected heritable trait(s). Non-limiting examples of types of preselected heritable traits that can be introduced into a target population using methods of the invention include: a trait that increases the population's robustness, a trait that increases likelihood of survival or an organism in the population, a trait that enhances a health characteristic of an organism in the population, a trait that increases resistance to disease of an organism in the population, etc. In some embodiments of the invention, heritable traits that may have a negative impact on a target population may be introduced and examples of types of such traits included, but are not limited to: a trait that decreases the population's robustness, a trait that decreases likelihood of survival of an organism in the population, a trait that negatively impacts a health characteristic of an organism in the population, a trait that decreases resistance to disease of an organism in the population, etc. A non-limiting example of a target population to which negatively impacting heritable traits may be added are malaria-carrying mosquitos, ticks, mice, rodents, pigs, etc.

After introduction of one or more preselected heritable traits into a population, the status of the introduced trait(s) in the population can be determined, which permits the success of the introduction to be evaluated. For example, though not intended to be limiting, relative numbers of organisms with and without the preselected heritable trait(s) can be determined and assessed over time as a measure of characteristics such as the distribution of the preselected heritable trait(s), changes in the size of the population over time, changes in the health of the population over time, etc. As used herein, the phrase “preselected heritable traits” may be used interchangeably with the phrase “preselected heritable phenotypic trait and/or preselected heritable genotypic trait” or the phrase “preselected heritable phenotypic and/or genotypic trait.”

As used herein the term “genetic characteristic” used in reference to an organism or species means an inherited trait, for example, trait that is inherited by an offspring or descendant from the organism's parent or an ancestor, respectively. A genetic characteristic introduced into a population of organisms can be a heritable trait that is introduced into one or more organism in the population. Non-limiting examples of changes in a genetic characteristic introduced into an organism or population of organism may be inclusion of a sterile-female heritable trait, a coat-color heritable trait, a fatherless-male heritable trait, and the like. The phrase: “genetic characteristic of a target population of organisms” may be used herein interchangeably with the phrase “heritable trait in the target population”. Thus, certain methods of the invention comprising altering a genetic characteristic of a target population of organisms, comprise introducing one or more preselected heritable traits into one or more of an organism and a plurality of organisms in a target population.

Embodiments of methods of the invention introduce into a population of organisms, one or more engineered organisms whose genomes comprise one or more genes or gene alleles that are responsible for a preselected heritable trait(s). Methods of the invention permit introduction in the population of the genes or gene alleles responsible for the preselected heritable trait(s), and thus are useful to bring new phenotypes into the population of organisms without adding new organisms with the phenotype into the population. In some embodiments of the invention, an engineered organism that delivers a new heritable trait into a population is an organism comprising a gene-drive. In certain embodiments of the invention, an organism released into the wild, or born into the wild comprises a gene drive that results in the presence of the preselected heritable trait in the population. For example, though not intended to be limiting, one or more of a preselected heritable trait is introduced into a population of organisms via “engineered” offspring comprising a gene drive and that are born to a host female that has been released into the population. Certain embodiments of methods of the invention can be used to introduce into a population of organisms, one or more engineered organisms comprising a gene drive, and the presence of the gene-drive organisms in the population deliver one or more heritable traits in the population by birth, without adding new organisms with the phenotype of the heritable trait into the population.

Methods of the invention permit introduction into a population of the genes or gene alleles responsible for the preselected heritable trait(s) using gene-drive methods and organisms comprising a gene drive, and thus certain embodiments of the invention are useful to bring new phenotypes into the population of organisms without adding new organisms with the phenotype into the population. Various methods to prepare and utilize a gene drive system, such as but not limited to a CRISPR-Cas9 system, to deliver one or more preselected heritable traits into an organism are known in the art, see for example, PCT Publication No.: PCT/US2017/031777, the contents of which is incorporated by referenced herein in its entirety. Gene-drive methods may be used in conjunction with certain embodiments of methods of the invention to deliver one or more preselected heritable traits into an organism and/or a target population of organisms.

In certain embodiments of the invention, following the introduction of the one or more preselected heritable traits into a population, organism in the population that exhibit the preselected heritable trait(s) are detected. The presence and/or relative numbers of organisms in the population that exhibit the one or more preselected heritable traits may also be determined and the results can be used in methods to assess gene flow, gene stability, and other features of the population of organisms. A population of organisms into which one or more preselected heritable traits are to be introduced, may be referred to herein as a “population of interest”, which is also referred to herein as a “target population”. An organism comprising one or more preselected heritable traits, such as an embryo, a fetus, an offspring, or a descendant of a host organism, may be referred to herein as an “engineered organism.”

In certain embodiments of the invention, an engineered organism has the genetics of organism of a population of interest, with one or more additional genes or gene alleles that are not present in the original population of interest. As a result of their nearly identical genetics, engineered and non-engineered organisms in a population are more likely to share characteristics such as but not limited to: fitness, reproductive success, and expected survival, compared to a population in which the genetics of the engineered and non-engineered organisms with less similar genetics. For example, an engineered organism produced by a host organism that did not have the same genetics of organisms of a target population may possess one or more characteristics sufficiently different from characteristics of the non-engineered members of the target population to reduce and/or prevent a level of compatibility and/or integration of the engineered and non-engineered organisms thereby prevent an accurate determination of gene flow and population changes. Certain embodiments of the invention include methods to maintain nearly identical genetics and shared characteristics in the engineered and non-engineered organisms in a population, and the methods increase the likelihood of successfully introducing the preselected heritable traits into a population, without significantly disrupting other characteristics of the population—thereby permitting use of methods of the invention to accurately assess gene flow and population changes. Methods of identifying and comparing genetics of organisms are routinely practiced in the art. Art-known methods can be used in conjunction with the teaching provided herein to maintain nearly identical genetics in engineered and non-engineered organisms and to use such organisms in methods of the invention.

The term “introducing” as used herein in reference to a preselected heritable trait and a target population refers to the preselected heritable trait becoming part of the genetics of the target population organisms. In certain embodiments of the invention, the introduction of the preselected heritable trait into the target population occurs begins following release of the impregnated females and subsequent birth of their engineered offspring. Engineered offspring of the released impregnated females possess genetics nearly identical to those in the target population and are capable of integrating into the target population, reproducing with members of the target population, and disseminating the preselected heritable trait(s) into offspring thereby expanding the presence of the preselected heritable trait in the genetics of the target population. Thus, using a method of the invention to introduce successfully one or more preselected heritable traits into a target population alters the genetics of the target population and results the presence of the one or more preselected heritable traits in organisms of the target population. Using methods of the invention, a total number of organisms with the altered genetics in a target population may increase following introduction of the preselected heritable trait(s).

In some embodiments of a method of the invention, introducing one or more preselected heritable trait(s) into a target population results in an increase—over time—in the number of organisms in the target population that exhibit the altered genetics. In certain embodiments of the invention, following release of one or more of the impregnated female organism, the total number of organisms in the target population that exhibit the altered genetics increases over time relative to the total number of organism in the target population that do not have the altered genetics. Use of near-identical genetics in engineered and non-engineered organisms in embodiments of the invention can ease behavioral integration of the engineered organisms into the target population and enhance reproductive success of the engineered organisms. Either or both of these factors may, over time, result in an increase in the relative number of organisms that exhibit the one or more preselected heritable trait(s) in the target population versus those that do not.

### Host Organisms

Certain aspects of the invention include methods for efficiently preparing in captivity large numbers of impregnated captive female organisms of a species. The term “impregnated captive female organism of a species” is used herein interchangeably with the term “host organism.” In some embodiments of the invention a host organism is prepared that includes one or more of a gene and gene allele that as a result, is included in the genome of offspring of the host organism.

An organism selected to be prepared as a host organism for use in methods of the invention may be selected, at least in part, because the organism has the same genetics as organism in a target population. In certain embodiments of the invention, an organism selected to be prepared as a host organism for use in methods of the invention is selected because it has been a member of the target population and thus may have one or more of knowledge of the local environment and social ties to other organisms in the population of organisms. Possession by a host organism of one or both of these features may enhance the host's ability to reproduce relative to a host organism that has not been a member of the population of interest.

In some embodiments of the invention a host organism has not been a member of the target population and is an organism that is: naive to the target population; transplanted into the target population from a different geographical area (even if of identical genetics); and/or transplanted in from a population of organisms that has had no contact with the target population (even if of identical genetics), etc.

Certain embodiments of the invention include obtaining an organism with the same genetics as organisms in a target population and preparing that organism as a host organism. Methods of the invention include preparing a host organism that is impregnated and into which one or more genetic elements have been introduced. An effect of the introduced genetic elements into the host organism is the inclusion in the genome of the host organism's offspring, the one or more genes or gene alleles not present in the genome of the host organism. In some aspects of the invention, a host organism has substantially the same genetics as the organisms in a target population, and an offspring of that prepared host organism also has the genetics of the target population, with the addition of the included one or more genes or gene alleles.

As used herein, the term “target population” is the population of organisms into which an impregnated female, or in some instances an engineered male organism, is released. Non-limiting examples of target populations include a wild population of the organism, an agricultural population of the organism, a human-managed population of the organism, a population of the organisms in a preserve, a population of the organisms in captivity; a zoo population of the organisms. In some embodiments of the invention, a target population is an endangered population of organisms. It will be understood that in some embodiments of the in invention a target population is the population from which the female organism was captured and in some embodiments of the invention the target population is a population of the same species of organism as the captured female, but it is a different population than the one from which the female organism was captured.

Various means can be used to introduce one or more gene alleles into offspring and descendants of a host organism of a species. In some embodiments of the invention, one or more genetic elements are introduced into a host organism. Introducing the genetic elements results in the offspring's genome include a gene or gene allele that is responsible for the offspring exhibiting a preselected heritable trait.

In some aspects of the invention one or more preselected heritable traits are introduced into a population of organisms of a species using methods that include introducing a genetic element obtained from one or more male organisms of the species that exhibit the preselected heritable trait(s), into one or a plurality of female organisms of the species, resulting in a host organism. Certain embodiments of the invention include release into a target population of one or a plurality of engineered male organisms prepared using a method of the invention. Some embodiments of the invention include release into a target population of one or a plurality of pregnant female organisms impregnated by an engineered male organism or impregnated in a manner that delivered the heritable trait into the target population. One or a plurality of host organism may be released into a population of interest. The term, “plurality” when used herein in reference to organisms, means more than one. In some embodiments of the invention a plurality of organisms is at least 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000 organisms, including all integers in the provided range. In some embodiments of the invention, a plurality is more than 1000 organisms.

Offspring and descendants of an impregnated captive female organism comprising an introduced genetic element exhibit the preselected heritable trait as a result of the introduction of the genetic element. The presence of one or more preselected heritable traits in an organism produced using a method of the invention, identifies the organism as a descendent of an impregnated captive female organism prepared using methods of the invention. Some embodiments of the invention include outcrossing to introduce selected genetic elements into a plurality of female captive organisms, wherein resulting descendants of the female captive organisms exhibit one or more preselected heritable traits. An embodiment of the invention may comprise introducing a genetic element into a captive female organism of a species via mating of the female with a captive male organism of the species. Certain embodiments of the invention comprise introducing a genetic element into a captive female organism of a species via a genetic engineering means.

### Heritable Traits

Some embodiments of the invention comprise including in a host organism one or more genetic elements that result in offspring of the host organism comprising a gene or gene allele the presence of which results in the offspring exhibiting a preselected heritable phenotypic and/or genotypic trait. An embodiment of the invention may comprise selecting one or more heritable phenotypic and/or heritable genotypic traits of organisms of a species. Heritable traits used in certain methods of the invention are also referred to herein as “preselected heritable traits” and a preselected heritable trait may be selected, at least in part, because the heritable trait is not present in organisms in a population of interest.

In some aspects of the invention, a preselected heritable phenotypic trait is a detectable phenotypic trait. A color of all or a portion of an organism's fur, referred to as “coat color” is a non-limiting example of a phenotypic trait that results from introducing a genetic element into a host organism of a species using a method of the invention. An introduced preselected heritable phenotypic trait, for example, coat color, is a coat color distinguishable from a coat color of an organism of the species that does not include the preselected heritable phenotypic trait. In certain embodiments of the invention, an organism of the species that does not include the introduced preselected heritable phenotypic trait is an organism having wild-type genetics. As a non-limiting example, an organism that has only wild-type genetics would exhibit a wild-type coat color and thus would be distinguishable from an engineered organism of the species that comprises wild-type genetics and includes the gene or gene allele resulting in the predetermined heritable coat color trait. Preselected heritable phenotypic traits that can introduced into a species using a method of the invention include, but are not limited to: disease resistance, altered behavioral responsiveness to aspects of the environment, altered behavioral responsiveness to human-controlled stimuli, altered interaction with another type of organism, altered dietary preference, recessive inviability, recessive sterility, development as a particular sex, resistance to tick-borne diseases, and resistant to ticks. Additional preselected heritable phenotypic traits that can be introduced into a species using a method of the invention are set forth elsewhere herein

In a non-limiting example, a target population may be a mouse population in which the mice exhibit coat color “A”. In such an instance, a heritable trait selected for introduction into that mouse target population might be a heritable coat color “X” that is visually distinguishable from the “A” coat color. A method of invention used in this circumstance, may include introducing the preselected “X” heritable trait into the target population and then detecting the presence and/or relative number of organisms in the target population that exhibit the heritable coat color “X”, compared to the number of organism in the target population that exhibit the “A” coat color. In this example, the presence of the “X” coat color in an organism identifies the organism as an engineered organism. Methods of the invention may also include determining a relative number of engineered organisms and non-engineered organisms that make up a targeted population following the introduction of one or more preselected heritable traits into the population.

In some embodiments of the invention, a genetic element introduced into a host organism results in one or a plurality of offspring and descendant organisms comprising a coat-color allele, which is a non-limiting example of a preselected heritable genotypic trait. In certain embodiments of the invention, a preselected heritable genotypic trait is a coat-color allele comprising a visible dominant (blaze) allele. In certain embodiments of the invention a preselected heritable genotypic trait is a coat-color allele comprising a visible recessive (Oca2^{P}, Tyr^{c-ch}) allele. Additional non-limiting examples of preselected heritable genotypic traits that may be included in an embodiment of the invention are: an antibody that confers disease resistance [e.g., see Buchthal, J. et al., (2018) Phil. Trans. R. Soc. B 374: 20180105, dx.doi.org/10.1098/rstb.2018.0105]; a genetic element that confers development as a male (e.g. Sry); a nuclease that prevents the development of daughters, and one or more additional genetic engineering systems that alter a gene resulting in one of the phenotypic traits listed elsewhere herein. In some embodiments of the invention, multiple copies of a genetic element are introduced into a host organism. In certain embodiments of the invention, one more copies of more than one genetic elements are introduced into a host organism.

In a non-limiting example, a preselected heritable trait is introduced into a wild population of P. leucopus, using embodiments of methods described herein. (e.g., see Example 1). A number of number of impregnated captive female P. leucopus organisms are prepared, each including one or more of a gene and a gene allele are included in the genome of offspring of the impregnated females. In some situations, the preselected heritable trait is introduced by delivering one or more selected genetic elements into a reproductive cell of each of the captive female organism. The introduction of the trait(s) may include delivery of one or more exogenous nucleic acid sequences into one or more reproductive cell(s) of the captive female organisms. The nucleic acid sequences may be delivered as part of a composition that comprises one or more selected genetic elements that are of interested to include in embryos of the captive female organisms. Such methods of the invention can be used to produce offspring and descendants of the captive females that exhibit one or more of the preselected heritable traits. The captive females into which the desired genetic elements are introduced are impregnated and may be released. Non-limiting examples of release situations are (i) release into the original population of the species from which a captive female was obtained and (ii) release into a wild population of the same species as the captive female that is not the original population from which the captive female was obtained. Offspring of the impregnated, released female organisms will include the preselected heritable trait(s) thus introducing the trait(s) into the population.

Following the release of the impregnated female organism into a population, methods of detecting the presence of the introduced preselected heritable trait(s) may be carried out. For example, though not intended to be limiting, a 1, 2, 3, 4, 5, 6, 7, 8, 9, 10, or more times after the introduction of the impregnated female organism, and/or their offspring, organisms in the population that exhibit the preselected heritable trait (also referred to as “engineered organisms”) can be detected. Numbers, geographic distribution, and other characteristics of engineered organisms in the population may be detected and assessed over time. Data on gene flow and dispersal of the engineered organism in the population can be collected and used to determine changes in population characteristics such as the relative number of engineered organisms compared to the number of non-engineered organisms in the population, the geographic location of engineered organisms, and other characteristics. Aspects of the invention permit delivery of genetic elements into organism that result in offspring of the organism that are resistant to tick-borne diseases and/or resistant to ticks.

### Daughterless-Males and Sterile Female Heritable Traits

In some embodiments of the invention, a preselected heritable trait impacts reproductive fitness of organism and/or that of a population into which the trait is introduced. Two non-limiting examples of such preselected heritable traits are daughterless-male and sterile-female heritable genotypic/phenotypic traits. These heritable traits can be generated by disrupting an activity of the X chromosome. For example, though not intended to be limiting, a daughterless-male heritable trait may be introduced into an organism and/or a population by engineering a male organism such that all offspring and descendants of the engineered male are male, including all offspring fathered by descendants of the engineered male. Some embodiments of the invention include a preselected daughterless-male heritable trait produced by engineering a male organism to include a system encoded on the Y chromosome that knocks out a critical sequence of an X-chromosome female-specific non-coding RNA gene and eliminates the non-coding RNA gene's activity, which is lethal to female embryos and results in only male offspring and descendants of the engineered male.

Some embodiments of the invention include a preselected sterile-daughter heritable trait produced by engineering a male organism to include a system encoded on the Y chromosome that knocks out a necessary sequence of an X-chromosomal or autosomal female-specific gene and eliminates the gene's activity, resulting in sterility of all female offspring and female descendants of the engineered male. As used herein, the term “fertility-associated gene” is used to indicate an X-chromosome female-specific gene that when its function is disrupted in a female organism, the female organism is sterile and/or has reduced fertility.

The term “introducing” as used herein in reference to a target population and a system encoded on the Y chromosome that knocks out a critical sequence of an X-chromosomal or autosomal female-specific gene and eliminates its activity, refers to the system encoded on the Y chromosome becoming part of the genetics of organisms in a target population. Two non-limiting examples of such systems that are encoded, are (1) a system encoded on the Y chromosome that knocks out a critical sequence of an X-chromosome female-specific non-coding RNA gene whose activity is required for female embryonic survival, (2) a system encoded on the Y chromosome that knocks out a critical sequence of X-chromosome female-specific genes whose activities are required for female fertility, and (3) a system encoded on the Y chromosome that produces a nuclease carried in the sperm that knocks out recessive female-specific fertility genes in the zygote or early embryo. Introduction of the system on the Y chromosome into the target population occurs following release of the engineered male organism and/or females impregnated with genetic material of the engineered male organism, and the birth of their engineered offspring. Offspring and descendants of an engineered male as described herein are capable to integrating into the target population, reproducing with members of the target population, and disseminating the system on the Y chromosome into their male offspring thereby expanding the presence of the daughterless-male heritable trait in the genetics of the target population. (See for example, U.S. Patent Application No. 62/912,323, the content of which is incorporated herein by reference in its entirety.)

A population of organisms into which engineered genetic material of the invention is introduced, for example by release of engineered daughterless male organism and/or release of impregnated female organisms carrying unborn engineered offspring, may be referred to herein as a “target population.” Successive generations of organisms in a target population following introduction of genetic material that disrupts activity of an X-chromosome female-specific non-coding RNA gene, or disrupts activity of an X-chromosome female-specific gene, will include increasing relative numbers of male organisms versus female organisms, and will result in fewer organism in a target population. As the ratio shifts, the presence of fewer female organisms and/or fewer fertile organism in the target population limits the reproductive capacity of the target population and over time reduces the number of organisms in the target population. After introducing one or more engineered male organism, or offspring thereof into a target population, the status of the gender ratio and/or numbers of organism in the population can be determined, thereby permitting assessment of the effectiveness of the introduction on the target population. Methods of the invention can be used to reduce numbers of organism in a target population but the reduction need not eliminate the population entirely. In addition, it will be understood that numbers of organism in a target population may be influenced by other events such as but not limited to environmental conditions, immigration of organisms into the target population, resource availability, etc.

Certain embodiments of methods of the invention comprise one or more of: (1) preselecting an X-chromosomal female-specific non-coding RNA gene, that disrupting the function of which in an embryo is lethal if the embryo is female and non-lethal if the embryo is male; (2) preselecting a Y chromosomal endogenous gene to engineer by introducing genetic elements/gene editing components, which may but need not be part of a translational fusion with the Y chromosome endogenous gene, wherein expression of the encoded gene editing components disrupts the function of the preselected X-chromosomal female-specific non-coding RNA gene; (3) introducing the preselected encoded genetic elements/gene editing components in a translational fusion to the preselected Y-chromosome endogenous gene in the germline of a male organism of a species to prepare an engineered daughterless male organism; (4) impregnating a female organism of the species with genetic material of the prepared engineered daughterless male organism, wherein male descendants of the impregnation are engineered daughterless males comprising the engineered Y-chromosomal endogenous gene that when expressed disrupts the preselected X-chromosomal female-specific non-coding RNA gene; and (5) releasing one or both of the impregnated female organism and the engineered daughterless male organism into a target population of the organism.

Similarly, some embodiments of methods of the invention comprise one or more of: (1) preselecting an X-chromosomal female-specific gene, that disrupting the function of which in sperm precursor cells results in sterility if the embryo is female and but not sterility if the embryo is male and therefore does not inherit the affect X-chromosome; (2) preselecting a Y chromosomal endogenous gene to engineer by introducing genetic elements/gene editing components, which may but need not be part of a translational fusion with the Y chromosome endogenous gene, wherein expression of the encoded gene editing components disrupts the function of the preselected X-chromosomal female-specific gene in sperm precursor cells; (3) introducing the preselected encoded genetic elements/gene editing components in a translational fusion to the preselected Y-chromosome endogenous gene in the germline of a male organism of a species to prepare an engineered sterile-daughter male organism; (4) impregnating a female organism of the species with genetic material of the prepared engineered daughterless male organism, wherein male descendants of the impregnation are engineered sterile-daughter males comprising the engineered Y-chromosomal endogenous gene that when expressed disrupts the preselected X-chromosomal female-specific gene; and (5) releasing one or both of the impregnated female organism and the engineered sterile-daughter male organism into a target population of the organism.

Similarly, some embodiments of methods of the invention comprise one or more of: (1) preselecting one or more female-specific genes required for fertility, that disrupting the function of which in an embryo results in sterility if the embryo is female but not sterility if the embryo is male; (2) preselecting a Y chromosomal endogenous gene to engineer by introducing genetic elements/gene editing components, which may but need not be part of a translational fusion with the Y chromosome endogenous gene, wherein expression of the encoded gene editing components leads to nuclease deposition in sperm that disrupts the function of the preselected female-specific fertility genes in the zygote or early embryo; (3) introducing the preselected encoded genetic elements/gene editing components in a translational fusion to the preselected Y-chromosome endogenous gene in the germline of a male organism of a species to prepare an engineered daughterless male organism; (4) impregnating a female organism of the species with genetic material of the prepared engineered daughterless male organism, wherein male descendants of the impregnation are engineered sterile-daughter males comprising the engineered Y-chromosomal endogenous gene that when expressed disrupts the preselected fertility genes gene; and (5) releasing one or both of the impregnated female organism and the engineered sterile-daughter male organism into a target population of the organism. Examples of female-specific fertility genes include: Afp, Il11ra1, Dlgap5, Juno, Kpna6, Cdc25b, Hsd17b1, FIGLA, Kpna7, Dppa3, NPM2, NOBOX, GDF9, OOEP, ZP3, ZAR1, Bmp15, NLRP5, PAD6, Filia, TLE6, PgR, Prl, Tnfaip6, Gja4, FshR, Fsh, Aaas, Adgrd1, Ambp, Antxr2, Camk2g, Cdc25b, Cpa2, Creld2, Fmn2, Igfbp7, Kpna2, Mir200b, Mir429, Pappa, Ptx3, Wnt6, Akr1c18, Cuzd1, Ddr1, Acls4, Pcytb1, Sat1, Sox17, Tkt1.

Some embodiments of methods of the invention include a gene-drive system. In a non-limiting example, an engineered organism comprising a gene drive system is prepared and/or used to introduce a daughterless-male heritable trait or a sterile-female heritable trait into a population of organism. In certain embodiments of the invention, an organism released into the wild, or born into the wild comprises a gene drive, which results in the introduction of one or more preselected heritable traits in the population. For example, though not intended to be limiting, one or more of a preselected heritable trait is introduced into a population of organisms via engineered offspring comprising a gene drive that are born to a host female that has been released into the population. Certain embodiments of methods of the invention may be used to introduce into a population of organisms, one or more engineered organisms comprising a gene drive, whose genomes comprise one or more genes or gene alleles that are responsible for genotypic changes that result in a preselected trait such as, but not limited, a daughterless-male trait or a sterile-female trait. Certain embodiments of methods of the invention comprise: (1) preparing a gene-drive system in an engineered organism and (2) using the engineered organism to introduce one or more genes or gene alleles responsible for one or more preselected heritable trait(s) into a target population by birth. These and certain other embodiments of the invention are useful to bring new phenotypes into a target population of organisms by birth, thus without adding new organisms with the phenotype into the target population.

### Engineered Organisms and Target Populations

Certain aspects of the invention include methods for preparing engineered male organisms of a species. A non-limiting example of an engineered organism prepared using a method of the invention and that can be used in some embodiments of methods of the invention is an engineered male organism prepared such that it includes a system that is integrated into the Y chromosome and that removes a sequence of an X-chromosome female-specific noncoding RNA gene that is necessary for its function. The system that has been introduced in the Y chromosome is lethal when present in an embryonic female of the organism, for example it is lethal if present in any female embryo generated with genetic material from an engineered male organism. The system that has been engineered into the male organism is inherited and included in the genome of the male offspring of the engineered male organism.

Another non-limiting example of an engineered organism prepared using a method of the invention and that can be used in certain embodiments of methods of the invention is an engineered male organism prepared such that it includes a system that is integrated into the Y chromosome and that removes a sequence of an X-chromosome female-specific gene that is necessary for the function of that gene. In some embodiments, the function of the gene is required for fertility in the female organism. The system that has been introduced in the Y chromosome results in infertile female when present in an offspring or descendant of the engineered male organism. The system that has been engineered into the male organism is inherited and included in the genome of the male offspring of the engineered male organism.

A male organism to be prepared as an engineered organism for use in methods of the invention may be selected, at least in part, because the male organism has the same genetics as organisms in a target population. This feature may enhance the male organism's ability to reproduce relative to an engineered male organism with genetics less similar to the target population organism. In some embodiments of the invention an engineered male organism has not been a member of the target population and is an organism that is: naive to the target population; transplanted into the target population from a different geographical area (even if of identical genetics); and/or transplanted in from a population of organisms that has had no contact with the target population (even if of identical genetics), etc.

Methods of the invention include preparing an engineered male organism and impregnating a female organism of the species with engineered genetic elements of the engineered male. Engineered genetic elements introduced into the Y chromosome a male organism using a method of the invention can be selected to result in the removal all or a portion of a sequence of for example, one of an X-chromosome female-specific non-coding RNA gene from the engineered male's X chromosome, which results in disruption of function of the non-coding RNA gene. Similarly, engineered genetic elements introduced into the Y chromosome a male organism using a method of the invention can be selected to result in the removal all or a portion of a sequence of for example, one of an X-chromosome female-specific gene from the engineered male's X chromosome, which results in disruption of function of the gene. In male embryos produced from the engineered male organism, a loss of function of the X-chromosome female-specific non-coding RNA gene or female-specific gene, may have minimal or no effect on the male embryo, but is lethal to female embryos or results in sterile female descendants, respectively. Receiving the disrupted X chromosome female-specific non-coding RNA gene from the engineered male is lethal to female embryos but is passed through subsequent male descendants of the engineered male organism. Receiving a disrupted X chromosome female-specific fertility-associated gene from the engineered male results in sterile or reduced fertility female offspring and descendants but is passed through subsequent male descendants of the engineered male organism. As used herein the term “at least a portion” when used in reference to a coat color, gene, sequence, or other item or characteristic means at least a part of the item or characteristic. Thus, in some embodiments, “at least a portion” refers to some but less than the whole item or characteristics and in certain embodiments “at least a portion” refers to all of the item or characteristic.

As used herein, the term “target population” is a population of organisms into which genetic elements prepared using methods of the invention, as in the non-limiting example of X-chromosomes comprising disrupted female-specific non-coding RNA gene that result in daughterless male organisms, is released. Non-limiting examples of target populations include: a wild population of the organism, an agricultural population of the organism, a human-managed population of the organism, a population of the organisms in a preserve, a population of the organisms in captivity; a zoo population of the organisms, a city population of the organisms, a rural population of the organism, etc. In some embodiments of the invention, a target population is a considered to be a pest and/or undesirable population of organisms.

It will be understood that in some embodiments of the invention a target population is the population from which the impregnated female organism was obtained prior to the impregnation, and in some embodiments of the invention the target population is a population of the same species of organism as the impregnated female, but is a different population than one from which the female organism was obtained.

Numbers, geographic distribution, and other characteristics of a target population into which one or more engineered organisms of the invention are included. Population numbers can be determined and changes assessed over time. Data on the efficacy of a release of one or a plurality of engineered males or impregnated females prepared using methods of the invention can be collected and assessed. Such an assessment can be used to aid in determining a number of engineered organisms of the invention to be released at one or more subsequent time points. Following release of one or more engineered daughterless male organism into a target population, methods of the invention may include determination of one or more changes in the ratio of female organisms to male organisms in the target population.

In some embodiments of the invention, an engineered organism comprises a gene-drive. In certain embodiments of the invention, an organism released into the wild, or born into the wild comprises a gene drive. In some embodiments of the invention, one or more of a preselected heritable phenotypic and/or genotypic trait is introduced into a population of organisms via “engineered” offspring that comprise a gene drive and are born to a host female that has been released into the population. Embodiments of methods of the invention introduce into a population of organisms, one or more engineered organisms comprising a gene drive, whose genomes comprise one or more genes or gene alleles that are responsible for a preselected heritable trait(s). Methods of the invention permit introduction in the population of the genes or gene alleles responsible for the preselected heritable trait(s) using gene-drive methods and organisms comprising a gene drive, and thus are useful to bring new phenotypes into the population of organisms without adding new organisms with the phenotype into the population.

### Genetic Elements

Certain aspects of the invention include methods of preparing impregnated captive female organisms of a species (also referred to herein as “host organisms”) that include embryos of fetal offspring that carry one or more selected genetic elements. The presence of the selected genetic element(s) in the embryos or fetuses of the host organism results in offspring and descendants that exhibit one or more preselected heritable traits. In some embodiments of the invention, one or more selected genetic elements are introduced into a reproductive cell of a host organism. In some aspects of the invention methods are provided that can be used to prepare a composition comprising genetic elements with which an exogenous nucleic acid sequence is delivered into a host reproductive cell. Such a composition may comprise one or more selected genetic elements of interest to introduce into a host organism. Embodiments of compositions that may be delivered into a host organism may be designed and constructed to include genetic elements described herein or other art-known genetic elements suitable to result inheritance of one or more preselected heritable phenotypic and/or genotypic traits by offspring and descendants of the host organism. Methods of the invention can be used to design and/or prepare one or more compositions that can be used to introduce genetic elements into a host organism. In some instances, embodiments of methods of the invention include design, construction, and/or use of one or more genetic elements, including, but not limited to: nucleic acid sequences, vector sequences, promoter sequences, and sequences encoding detectable labels, such as but not limited to fluorescent labels.

Selecting one or more genetic elements to introduce into a captive female organism may include selecting one or more of a gene sequence, an allele, a delivery agent, a promoter sequence, a spacer sequence, a detectable label such as fluorescent detectable label, etc. that are appropriate to result in the exhibition of one or more preselected heritable phenotypic traits and heritable genotypic traits in a descendant of the impregnated captive female organism. Certain aspects of the invention include use of art-known design and construction methods with which to include one or more genetic elements in the impregnated captive female organism. In some embodiments of the invention, genetic elements used in methods of the invention are delivered using artificial means, a non-limiting example of which is use of a delivery vector.

Some embodiments of the invention comprise introducing a genetic element into a captive female organism of a species via mating of the female with a captive male organism of the species. The term “mating” as used herein, means the action of organisms coming together to breed, for example, copulation and the transfer of genetic elements from an organism of one sex to an organism of the opposite sex. In certain embodiments of the invention a male organism of a species comprises a genetic element that is introduced into a female organism of the species when the two organism mate and the presence of the introduced genetic element in the impregnated female organism results in offspring and descendants of the female organism exhibiting one or more preselected heritable phenotypic traits and heritable genotypic traits. In some embodiments of the invention, the genetic element introduced into the female organism is selected at least in part because its introduction in the female results in the exhibition of a heritable phenotypic trait and/or a heritable genotypic trait of interest in a descendent of the impregnated female organism. In some embodiments of the invention, a genetic element is delivered into a reproductive cell of the female of the organism. An effect of a genetic element of the invention manifests in offspring and descendants of the impregnated captive female organism. The one or more preselected heritable phenotypic trait and/or preselected heritable genotypic trait is exhibited in offspring and descendants of the impregnated captive female organism.

In other embodiments of the invention, the genetic elements may be introduced into a captive female organism of the species through artificial insemination. The term “artificial insemination” as used herein, means the introduction of sperm carrying a genetic element into a female organism of the species. Methods that can be used to obtain sperm for use in methods of the invention, including but not limited to artificial insemination methods, are known in the art.

Certain embodiments of methods of the invention include one or more of: (1) preselecting one or more heritable traits of interest to be exhibited in an organism of a species; (2) selecting one or more genes and/or gene alleles that when expressed in an organism of the species result in the organism exhibiting the preselected heritable trait(s); (3) introducing one or more genetic elements into a host organism, wherein the introduction results in offspring of the host organism that exhibit the one or more selected genes and/or alleles; (4) releasing the host organism into a population of organism of interest; which results in the presence of the preselected heritable traits in one or more offspring of the host organism in the population of interest. In some embodiments, the host organism is a female organism captured from the target population, and the captured female organism is impregnated in conjunction with the introduction into the female organism of genetic material that results in the preselected heritable trait(s) in offspring of the impregnated female organism. The impregnated host organism may then be released back into a population of the organisms. Following release of an impregnated female prepared in a manner to include genetic material that results in “engineered” offspring that exhibit the one or more preselected heritable traits, the released female gives birth to offspring in the target population. Offspring of the released female exhibit one or more of the preselected heritable traits.

The term “introduction by birth” as used herein, means introduction of one or more preselected heritable traits into a target population using a method of the invention comprising release of an impregnated female host into the target population and the subsequent birth of offspring of the pregnancy when the released female is in the target population. In contrast to methods that include introduction by birth, another method of introducing one or more preselected heritable traits into a target population is by direct releasing an organism that exhibits the one or more preselected heritable traits into a target population.

Genetic elements, selected genes, and selected, gene alleles that can be used in methods of the invention may include a gene and/or gene allele sequence that when present in the genome of an organism, the organism exhibits a preselected heritable phenotypic and/or genotypic trait. Methods of the invention include selecting a heritable trait that can be detected and monitored in organisms in a population. Art-known means can be used to help select and introduce one or more genetic elements into a host organism, the introduction of which results in descendants of the host organism exhibiting the preselected heritable trait. Routine genetic engineering means can be used in embodiments of methods of the invention to introduce one or more genetic elements into a host organism such that descendants of the host organism exhibit the preselected heritable trait. As used herein, genetic elements included in embodiments of the invention may be independently selected.

A composition of the invention may comprise one or more genetic elements that when introduced into a host organism, impact the phenotype and genotype of offspring of the host organism. Thus the genetics of a host organism may differ from the genetics of an offspring of the host organism, in that introduction of one or more genetic elements into the host organism results in a selected gene and/or gene allele in the offspring genome that is not present in the genome of the host organism. Components of compositions of the invention, for example genetic elements, may be delivered into a host organism using standard genetic engineering techniques, mating, or other suitable methods. In some embodiments of the invention, introducing a preselected heritable trait into a population of organism may comprise a targeted recombination method. In certain aspects of the invention, vectors are used to deliver a composition of the invention into a host organism.

### Genetic Elements and Gene Editing Components

Certain aspects of the invention include methods of preparing one or more engineered daughterless or sterile-daughter male organisms of a species. Embodiments of methods of the invention may include delivering gene-editing components, which may also be referred to herein as “genetic elements” and/or “genetic material” into the germline of the engineered male organism of the species. Delivery of gene editing components can be performed using art-known methods. For example, using vectors, etc. and other delivery means. Some embodiments of the invention include methods for designing and/or preparing one or more compositions that can be used to introduce genetic elements into an organism. In some instances, embodiments of methods of the invention include design, construction, and/or use of one or more genetic elements, including, but not limited to: nucleic acid sequences, vector sequences, promoter sequences, and sequences encoding detectable labels, such as but not limited to fluorescent labels. Gene editing components and means of creating engineered organisms using gene-editing components are known and routinely used in the art.

Selecting one or more gene editing components to introduce into a male organism to prepare the engineered male organism, may include selecting one or more of a gene sequence, an allele, a guide RNA sequence, a nuclease, a translation fusion molecule, a delivery agent, a promoter sequence, a spacer sequence, a detectable label such as fluorescent detectable label, etc. that are appropriate to result in the engineered male organism. In some embodiments, the components are introduced to the male organism's germline. Certain aspects of the invention include use of art-known design and construction methods with which to include one or more genetic elements in an engineered male organism in a manner that they can be delivered to a female organism of the species. Some embodiments of the invention comprise introducing a Y chromosomal engineered gene from an engineered male into a female organism of a species via mating of the female with the engineered male of the invention. The term “mating” as used herein, means the action of organisms coming together to breed, for example, copulation and the transfer of genetic elements from the engineered male to the female organism of the species. In addition to mating, the presence of the genetic elements components in a germline cell can permit transfer of the Y-chromosomal engineered gene using methods such as, but not limited to artificial insemination and in vitro fertilization—to impregnate a female of the organism with the engineered genetic elements of the invention. In some embodiments of the invention, genetic elements used in methods of the invention are delivered using an artificial means, a non-limiting example of which is use of a delivery vector.

Routine genetic engineering and gene editing methods can be used in conjunction with methods of the invention set forth herein to select and introduce one or more genetic elements in and prepare an engineered daughterless male organism, whose descendants are all engineered daughterless males. As used herein, the term “vector” used in reference to delivery of compositions of the invention refers to a polynucleotide molecule capable of transporting between different genetic environments another nucleic acid to which it has been operatively linked. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked may be referred to herein as “expression vectors.” Other useful vectors, include, but are not limited to viruses such as lentiviruses, retroviruses, adenoviruses, and phages. Vectors useful in some methods of the invention can genetically insert one or more genetic elements into a dividing or a non-dividing cell and can be used to introduce one or more compositions of the invention that comprise genetic elements into an in vivo or in vitro cell. In certain embodiments of the invention, encoded gene editing components are delivered into a germline cell of an engineered male organism and when expressed in the cell the gene editing components function to disrupt a preselected X-chromosomal female-specific non-coding RNA gene in the cell. In certain embodiments of the invention expression of one or more of the genetic components delivered into a host organism are expressed in a host cell and the effect of the activity of the genetic components is passed by the host organism to the host organism's offspring and into later generations of organism arising from the host organism.

Vectors useful in methods of the invention may include sequences including, but not limited to one or more of a: promoter sequence; recombinase encoding sequence; enhancer sequence; additional components used in targeted recombination methods; components suitable for use in gene editing methods, non-limiting examples of which are: guide nucleic acid sequences, guide RNA sequences, sgRNA sequences, promoter sequences, DNA-binding protein encoding sequences, nuclease enzyme encoding sequences, RNA-guided nuclease enzyme encoding sequences, programmable nuclease enzyme encoding sequences, programmable base editor enzyme encoding sequences, RNA-guided base editor enzyme encoding sequences, CRISPR system components, CRISPR/Cas9 system components, 2A “ribosomal skipping” peptides; and detectable label encoding sequences, etc. Methods of the invention can be used to design and construct vectors comprising genetic elements that when delivered in a method of preparing an engineered male organism, result in the inclusion of engineered genetic materials in male descendants of the engineered male organism.

Other useful vectors, include, but are not limited to viruses such as lentiviruses, retroviruses, adenoviruses, and phages. Vectors useful in some methods of the invention can genetically insert one or more genetic elements into a dividing or a non-dividing cell and can be used to introduce one or more compositions of the invention that comprise genetic elements into an in vivo or in vitro cell. Expression vectors and methods of their use are well known in the gene-editing arts. Promoters that may be used in methods and vectors of the invention include, but are not limited to, cell-specific promoters or general promoters. Methods for selecting and using cell-specific promoters and general promoters are well known in the gene editing and recombinant arts.

In some embodiments of a method of the invention, one or more promoters is encoded upstream of the one or more encoded guide RNAs. In some such instances, the polymerase(s) may enhance expression of the one or more guide RNAs compared to the expression of the one or more guide RNAs in the absence of the encoded promoter(s). In some embodiments of the invention, the promoter is a polymerase III promoter, examples of which include, but are not limited to a U6 promoter, a 7SK promoter, an H1 promoter, and a tRNA promoter. A promoter included in some embodiments of the invention is a synthetic promoter. A non-limiting example of which is a synthetic polymerase III promoter. In some embodiments, the one or more encoded promoters, such as polymerase III promoters, are independently selected. As used herein, the term “independently selected” in reference to components such as, but not limited to genetic elements, promoters, etc., means if two or more of a category of components are selected, they are chosen for inclusion independent of the others in that category of components. For example, in an embodiment of the invention comprising two or more promoters, the promoters may be independently selected, and as a result, in some embodiments all of the selected promoters are the same, in some embodiments all of the selected promoters are different from each other, and in some embodiments, when there are more than two independently selected promoters two or more of the promoters are the same as each other and different from one or more other promoters.

In certain embodiments of the invention, there is one polymerase III promoter, and in some embodiments, there are one, two three, four, five, six, seven, eight, or more independently selected encoded promoters. In some embodiments of the invention, there are one, two three, four, five, six, seven, eight, or more independently selected encoded promoter polymerase III promoters. In embodiments in which there are two or more promoters, they may be independently selected for inclusion. Thus, in some embodiments, all of the promoters are the same as each other; in some embodiments, two or more of the promoters are the same as each other; and in certain embodiments each of the promoters is different from the other promoters. A promoter included in some embodiments of the invention is a promoter native to the organism species, for example, the promoter may be native to the species of the engineered male organism. A promoter included in some embodiments of the invention is a promoter that is not native to the organism species, for example, the promoter may be a promoter that is not native to the species of the engineered male organism.

### Y-Chromosomal Genes and Fusions

As described herein, a Y chromosomal endogenous gene can be selected for inclusion in a translational fusion. As described elsewhere herein, in some embodiments of the invention a translational fusion to an endogenous gene comprises one or more sequences encoding one, two, three, or more components of a gene editing system such as, but not limited to: a guide RNA, a nuclease, etc. In certain embodiments of the invention, a nuclease is introduced but not as part of a translational fusion to the endogenous gene. Non-limiting examples of Y-chromosomal genes that may be included in a translational fusion and used in a method of the invention are Eif2s3y, Zfy, and Ddx3y. A non-limiting example of a translational fusion to the Y-chromosomal endogenous gene that can be used in certain embodiments of methods of the invention is an N-terminal 2A-fusion to the Y-chromosomal endogenous gene.

In some embodiments of the invention, methods and compositions are provided that can be used to increase translation of an N-terminal 2A-fusion to the endogenous gene. It has been recognized that an N-terminal 2A-fusion to a gene may exhibit a reduced level of translation of the gene. Certain aspects of the invention include methods and compositions to lessen the reduction of transcription of the gene of an N-terminal 2A-fusion. Such methods of the invention may include preparing an N-terminal 2A-fusion to a gene, wherein the N-terminal 2A-fusion comprises a modified Kozak sequence. Inclusion of the modified Kozak sequence can increase the level of translation of the gene compared to the level of translation of the gene when the N-terminal 2A-fusion does not comprise the modified Kozak sequence. An N-terminal 2A-fusion that includes a modified Kozak sequence may also include a sequence that encodes a nuclease and one or more sequences encoding guide RNAs. A gene included in the N-terminal A2-fusion may be present in an organism and in some embodiments, the gene is an endogenous gene that naturally occurs in the organism. Certain embodiments of the invention include in a translational fusion an endogenous gene that is expressed in the germline of males of the organism. An example of an endogenous gene that may be used in some embodiments of the invention is Y-chromosomal gene, non-limiting examples of which are: Eif2s3y, Zfy, and Ddx3y.

Another method to increase translation of an N-terminal 2A-fusion to a gene is provided in certain embodiments of the invention in which an N-terminal 2A-fusion to a gene is prepared and the preparation comprises inserting a plurality of introns into the coding region of the gene. As a result of the method, the inserted plurality of introns increases the level of translation of the gene compared to the level of translation of the gene in the absence of the inserted plurality of introns. An N-terminal 2A-fusion comprising an inserted plurality of introns in the coding region of the gene may also include a sequence that encodes a nuclease and one or more sequences encoding guide RNAs. A gene into which is inserted the plurality of introns and is included in the N-terminal A2-fusion may be present in an organism and in some embodiments the gene is an endogenous gene that naturally occurs in the organism. Certain embodiments of the invention include in a translational fusion an endogenous gene comprising the inserted plurality of introns, wherein the gene is expressed in the germline of males of the organism. An example of an endogenous gene that may be used in some embodiments of the invention is Y-chromosomal gene, non-limiting examples of which are: Eif2s3y, Zfy, and Ddx3y. An intron inserted into the gene may be a synthetic codon, an intron that is native to the organism in which the translational fusion is present.

In some embodiments of the invention, a translational fusion to a gene can be prepared that comprises a modified Kozak sequence and comprises a plurality of introns inserted into a coding region of the gene.

### X-Chromosomal Genes

A non-limiting example of an X-chromosomal female-specific non-coding RNA gene that may be included in a method of the invention is an X-inactive specific transcript (Xist) gene. Loss of Xist function has no known effects in male organism, such as mice, nor in daughters who inherit a dysfunctional copy from their mother. However, loss of function mutations in the paternal copy of Xist are selectively lethal in embryonic daughters. In a non-limiting example, disrupted function of the Xist gene, for example loss of function of a paternal copy of Xist is lethal to female mouse embryos at embryonic day 8.5 due to failed X-inactivation in the trophoblast [Marahrens et al. (1997) Genes & Development Jan 15; 11(2):156-66; Wutz, A. et al., (2002) Nat Genet. February; 30(2):167-74; and Hoki et al. (2009) Development January; 136(1):139-46; the content of each of which is incorporated by reference herein in its entirety].

Certain embodiments of methods of the invention include gene editing to delete all or a portion of Xist exon 1 that encodes repeated stem-loop motifs. A minimal number of encoded repeated stem-loop motifs are strictly required for X-inactivation mammals such as mice (see for example: Wutz, A. et al., (2002) Nature Genetics February; 30(2):167-74; and Hoki et al. (2009) Development January; 136(1):139-46; the content of each of which is incorporated by reference herein in its entirety]. Certain embodiments of methods of the invention that include expressing an RNA-guided nuclease and a plurality of guide RNAs that target sequences flanking or distributed throughout this region, yield a targeted deletion and loss of Xist function. An alternative method comprises use of a targeted recombinase, such as but not limited to an RNA-targeted recombinase, to excise the relevant region of the non-coding gene.

The Xist gene sequence is highly conserved across mammals, and in certain embodiments methods of the invention are used to prepare engineered daughterless male organisms of the genus Rattus, genus Mus, genus Sus, genus Felis or genus Canis. It will be understood that engineered daughterless male organisms of other genera of mammals may also be prepared using methods of the invention. In some embodiments of the invention, methods of the invention are used to prepare engineered daughterless male organisms of mammalian invasive species and pests [Loda, A., & Heard, E. (2019) PLoS Genetics 15 (9): e1008333. eCollection, the content of which is incorporated herein by reference in its entirety].

A Y-chromosome-linked system of the invention can be used to disrupt Xist functionality on the X chromosome. Certain embodiments of methods of the invention include encoding a RNA-guided nuclease and multiple guide RNAs targeting sequences flanking this region, such that nuclease activity occurs in male germline cells prior to spermiogenesis. Cutting multiple sites in flanking regions and within the relevant region leads to targeted deletions when repaired by non-homologous end-joining. In certain embodiments of the invention, the engineered cells include germline cells, which minimizes potential fitness costs of nuclease expression.

In some embodiments of the invention, one or more nucleases are encoded as an N-terminal 2A-peptide fusion [Trichas, G., et al., (2008) BMC Biology 6 (September): 40; Liu, Z., et al. Sci Rep 7, 2193 (2017) doi:10.1038/s41598-017-02460-2; the content of each of which is incorporated herein by reference in its entirety] to a Y chromosome-encoded protein expressed in the male germline. As a non-limiting example, the protein Eif2s3y is expressed throughout male germline development, which allows ample time for the nuclease to disrupt Xist [Mazeyrat et al. (2001) Nature Genetics September; 29(1):49-53; the content of which is incorporated herein by reference in its entirety]. Methods of the invention may include a 2A peptide fusion to a Y-chromosomal gene and such embodiments of methods of the invention can result in reliable expression in the male germline [Mazeyrat, S., et al., (2001) Nature Genetics 29 (1): 49-53; Armoskus, C., et al. (2014) Brain Research 1562 (May): 23-38; Huby, R. D. J. et al., (2014), PloS one, 9(12):e115792; Li, Na, et al., (2016) Oncotarget 7 (10): 11321-31]. To compensate for the reduced expression of the Y-chromosomal gene resulting from inefficient translational re-initiation after the 2A peptide, in some embodiments of methods of the invention, the Kozak sequence controlling translational initiation is strengthened to the consensus sequence, and/or introns are inserted into one or more of the nuclease gene sequence to enhance transcription. Another non-limiting example of a Y-chromosomal gene expressed in the male germline that may be used in methods of the invention is Zfy2, which provides a narrow burst of expression during late meiosis. Certain embodiments of the invention preparing an engineered male organism comprises introducing one or more nucleases that are not part of a translational fusion to the Y-chromosomal endogenous gene. For example, in some embodiments of the invention, one or more nucleases introduced in a method of the invention may be encoded with its own expression signals away from existing endogenous Y chromosomal genes, although this may result in reduced activity. Embodiments of methods of the invention may include any method of expression that results in sufficient expression of the nuclease to achieve Xist deletion in the male germline. Some embodiments of the invention may include steps to reduce expression or confer tissue-specific expression in the event a practitioner desires to minimize potential fitness costs resulting from expression of the nuclease in all cells rather than only germline cells.

### Variants

Components of a composition of the invention may include sequences described herein, art-known sequences and may include functional variants of such sequences. A variant nucleic acid sequence may encode a variant polypeptide that includes one or more of a deletion, point mutation, truncation, amino acid substitution, and addition of an amino acid or non-amino acid moieties, as compared to its parent polypeptide. Modifications of a polypeptide of the invention (a non-limiting example of which is an expressed genetic element) may be made by modification of the nucleic acid sequence that encodes the polypeptide. The terms “protein” and “polypeptide” are used interchangeably herein as are the terms “polynucleotide” and “nucleic acid sequence.” A nucleic acid sequence may comprise genetic element including, but not limited to RNA, DNA, mRNA, cDNA, etc. As used herein with respect to polypeptides, proteins, or fragments thereof, and polynucleotides that encode such polypeptides the term “exogenous” means the one that has been introduced into a cell, cell line, organism, or organism strain and is not naturally present in the wild-type background of the cell or organism strain.

As used herein the term “variant” in reference to a polynucleotide or polypeptide sequence refers to a change of one, two three, four, five, six, seven, eight, nine, ten, or more nucleic acids or amino acids, respectively, in the sequence as compared to the corresponding parent sequence. For example, though not intended to be limiting, a variant gene allele sequence may be identical to that of its parent gene allele sequence except that it includes 1, 2, 3, 4, 5, 6, 7, 8, 9, 10, or more nucleic acid substitutions, deletions, insertions, or combinations thereof, and thus is a variant of the parent gene allele. Certain methods of the invention for designing and constructing compositions for use in methods of the invention include methods to prepare functional variants of genetic element components of compositions such as encoding gene sequences, recombinase sequences, or other sequences used in methods of the invention.

Methods of the invention provide means to test for activity and function of variant sequences and to determine whether a variant is a functional variant and is suitable for inclusion in a composition and/or method of the invention. Suitability can, in some aspects of methods of the invention, be based on one or more characteristics such as: expression; a resulting phenotypic trait, stability of the sequence change in offspring and descendants of a host organism, survival of a host organism and/or its descendants, etc. A functional polynucleotides that may be used in embodiments of methods and compositions of the invention may be nucleic acid sequences that have at least 85%, 86%, 87%, 88%, 89%, 90%, 91%, 92%, 93%, 94%, 95%, 96%, 97%, 98%, or 99% identity to their parent nucleic acid sequence.

Art-known methods can be used to assess relative sequence identity between two amino acid or nucleic acid sequences. For example, two sequences may be aligned for optimal comparison purposes, and the amino acid residues or nucleic acids at corresponding positions can be compared. When a position in one sequence is occupied by the same amino acid residue, or nucleic acid as the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment can be performed using any one of a number of well-known computer algorithms designed and used in the art for such a purpose. It will be understood that a variant polypeptide or polynucleotide sequence may be shorter or longer than their parent polypeptide and polynucleotide sequence, respectively. The term “identity” as used herein in reference to comparisons between sequences may also be referred to as “homology”.

### Release

In some aspects of the invention, one or a plurality of host organisms of a species are prepared as set forth in an embodiment of the invention such that offspring of the impregnated captive female organism(s) exhibit a preselected heritable trait. Host organisms may be released into a population of interest and give birth to offspring that exhibit the preselected heritable trait. One or more offspring of the released host organism may be impregnated by mating with a male organism in the population and the preselected heritable trait exhibited by offspring resulting from the mating. This process may occur 1, 2, 3, 4, 5, 6, 7, 8, 9, 10, or more times thereby producing organisms that are descendants of a host organism and exhibit the preselected heritable traits. By reproducing in the organism population, offspring and descendants of a host organism establish and/or spread the preselected heritable trait(s) in the population. One or more of (1) genotyping to detect the presence or absence of one or more genes in an organism and (2) detecting the presence or absence of a phenotypic trait in an organism may be used to identify new male carriers of preselected heritable trait(s) of interest, and these male carriers can be used (non-limiting examples of which are for mating and to obtain genetic material of interest) to repeat the process of preparing a host organism.

In some embodiments of the invention one or a plurality of impregnated captive female organisms (host organisms) are released into a population of the organisms. In some instances, the host organism are released into a population of organisms prior to giving birth to her gestating offspring. In certain embodiments of the invention, a host organism may be retained in captivity until after birth of her offspring, and the offspring are later released into the population of organisms. A population of interest may be a population that is in the wild or may be a captive population of the organism. In some embodiments of the invention, a population of organisms is a population not in captivity. In some instances, a population of organisms is a controlled population, for example a captive, lab-maintained population of organisms. In other instances, a population of organisms is a wild population or is a domesticated population of organisms.

Multiple releases of host organism into a population are contemplated in certain aspects of the invention. Some embodiments of the invention comprise 1, 2, 3, 4, 5, 6, 7, 8, 9, 10, or more releases of one or a plurality of impregnated captive female organisms prepared using a method of the invention into a population of organisms of the species. In some embodiments of the invention a plurality of impregnated captive female organisms of a species are released into a population of organisms of the species at 1, 2, 3, 4, 5, 6, 7, 8, or more geographic locations. In some embodiments of the invention, there are 1, 2, 3, 4, 5, 6, 7, 8, or more releases of one or a plurality of impregnated captive female organisms of a species into a population of organisms of the species and the number of impregnated captive female organisms in each release may be the same or may include different numbers of impregnated captive female organisms. For example, a first release may include 20 impregnated captive female organisms and a subsequent release may include 50 impregnated captive female organisms. In certain embodiments of the invention factors such as: (1) the number of impregnated captive female organisms released into a population, (2) the geographic release location(s), (3) the total number of impregnated captive female organisms released into a population of the organisms, (4) the timing of one or more releases of impregnated captive female organisms into a population, and/or other release characteristics are determined based on factors including but not limited to: geographic area of the population, topography of the environment that includes the population, population size, geographic range of the population, behavior of organisms of the species, and density of the population of organisms.

### Detecting and Monitoring

Methods of the invention, in some embodiments, may include monitoring one or more preselected phenotypic heritable traits in a population over time. Certain embodiments of the invention include one or more of (1) determining the presence of one or more of a preselected phenotypic heritable traits in the population of interest at one or more time points and (2) determining in the population of interest one or more changes in the relative number of organisms that exhibit the preselected heritable phenotypic trait compared to the number of organisms that do not exhibit the preselected heritable phenotypic trait. In non-limiting examples, the presence of a preselected heritable coat color may be monitored over time and a change in a relative number of organisms that exhibit the preselected heritable coat color may be determined over time, etc. Some embodiments of methods of the invention include use of art-known methods to detect and determine actual and/or estimated numbers of organism in a captive population and/or a wild population of the organisms. Methods of the invention can be used to assess a population into which a preselected heritable trait has been introduced. A non-limiting example of a population characteristic that may be determined using a method of the invention is a ratio of the number of engineered organisms versus non-engineered organisms in a population following the introduction of a preselected heritable trait into a population of the organisms. Some embodiments of the invention include determining the presence or absence of a preselected phenotypic heritable trait in a population and/or determining a relative number of engineered organism and/or non-engineered organism in the total population. It will be understood that identifying an organism as an engineered organism produced using a method of the invention may comprise detecting that the organism exhibits a preselected heritable phenotypic trait. Similarly, a method of identifying an organism as an engineered organism produced using a method of the invention may comprise detecting that the organism's genetics comprises a preselected heritable genotypic trait. It will be understood that identifying an organism that exhibits a preselected heritable phenotypic trait also confirms that the organism carries the corresponding preselected heritable genotypic trait. Results obtained using detection and determination methods of the invention can be used to assess and/or identify the presence, absence, and/or spread of a heritable genotype trait (e.g., gene allele) responsible for an exhibited preselected heritable phenotypic trait. In some aspects of the invention, genetic elements that are delivered into an organism result in offspring of the organism that exhibit one or both of the preselected phenotypic traits of resistance to tick-borne diseases and/or resistant to ticks.

Methods of the invention comprising detecting or determining the number of engineered organisms in a targeted population can be performed once or more than once. It will be understood that the number of engineered organisms determined in an initial population before the introduction of one or more engineered heritable traits may be zero. Embodiments of methods of the invention may include determining a relative number of engineered organism in a population before an introduction of one or more engineered heritable traits and/or one or more times after an introduction of one or more engineered heritable traits into the population of organisms. Two or more determinations of the relative number of engineered organisms in the total number of organisms in a population may, in methods of the invention, be used in assessing population characteristics, such as but not limited to: changes in the proportion of the engineered organisms in the overall population, temporal and/or geographic dispersal of engineered organisms through the population, stability of the one or more preselected heritable traits in the population, changes in one or more other heritable traits in the population over time, changes in the geographic distribution of organisms in the population, etc.

Some embodiments of the invention include monitoring gene flow and population changes by assessing the presence and/or spread of preselected heritable traits in a population of organism of a species into which an impregnated captive female organism of the species prepared using methods of the invention was released. Offspring and descendants of the released impregnated captive female organisms exhibit one or more of a preselected heritable phenotypic trait and/or heritable genotypic trait and can thereby be distinguished from organisms of the species that do not exhibit the preselected trait(s). Embodiments of the invention comprising introducing genetic changes, monitoring gene flow, and determining changes in the recipient population, in part, may include art-known methods. See for example: Berry, R. et al., (1991) J. of Zoology 225:615-632; Jones, C. S. et al., (1995) Proc. of the Royal Society of London, Series B: Biological Sciences 260:251-256; and Scriven, P. J. (1992) J. of Zoology 227:493-502), each of which is incorporated herein by reference in its entirety.

Certain embodiments of methods of the invention may be used to assess gene flow and genetic changes in a population of organisms. As a non-limiting example, one or a plurality of impregnated female organisms is prepared using methods of the invention that introduce genetic elements to the female organisms and as a result descendants of the impregnated females will exhibit one or more preselected heritable phenotypic traits and/or preselected heritable genotypic traits. The one or plurality of the impregnated female organisms are released into a population of organisms of the same species as the females and after the release, the resulting population of organism is assessed to determine a relative number of organisms in the population that include the one or more preselected heritable traits as compared to the total number of organism in the population. Such relative numbers in a population of organisms can be determined 1, 2, 3, 4, 5, 6, 7, 8, 9, 10, 20, 30, or more times. Results obtained from determinations at different times may be compared to detect changes in gene flow and genome changes in the organism population over time. Determinations of the total number of organisms in a population, and the total number of wild-type organism in the population can also be compared to the initial number of organisms (all would be wild-type) determined to be in the organism population before the release of the impregnated female organisms. A change in the total population over time can be determined using routine, art-known population-measuring methods. A ratio of the number of organisms in the population that exhibit one or more of the preselected heritable phenotypic traits and/or preselected heritable genotypic traits compared to the number of organism in the population that do not, e.g., wild-type organisms, can be determined once or a plurality of times and the results obtained may be used to assess one or more factors such as, but not limited to: population increases, population decreases, gene flow, and changes in genome characteristics in part or all of the organism population. Changes in the population numbers, proportion of organisms with one or more of the preselected heritable traits compared to wild-type organisms, as well as other organism and population changes can be detected by obtaining information such as: population measurements, phenotype determinations, and genotype determinations at two or more different times and comparing the results.

A time at which detecting one or more of the preselected heritable phenotypic traits and/or preselected heritable genotypic traits may be done is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 40 weeks, 60 weeks, 80 weeks, 100 weeks, or 200 weeks after a release of impregnated captive female organisms, or their offspring. It will be understood that the time interval between any two detection times is independently selected and can, but need not, be equal.

The term “detecting” as used herein in reference to the preselected heritable phenotypic traits and/or preselected heritable genotypic traits may mean any means of measuring a characteristic of the traits. As a non-limiting example, detecting may comprise visually observing an organism and determining the presence or absence of one or more of preselected heritable phenotypic traits and/or preselected heritable genotypic traits introduce into the population of the observed organism.

Detecting one or more preselected heritable phenotypic traits and/or preselected heritable genotypic traits in a population of organisms may be performed one time, two times, or more than two times, with the detections performed at regular time intervals. As used herein, the term “regular time intervals” means approximately equal time elapses between each two consecutive detections of the preselected heritable traits in the population. Regular detection intervals are not required, and in some embodiments of the invention comprising multiple detections and/or determinations of the one or more preselected heritable phenotypic traits and/or preselected heritable genotypic traits time intervals between the detections, are not regular time intervals.

### Organisms and Cells

One or more methods of the invention for designing, preparing, and using compositions that introduce a preselected heritable trait into a population of organisms of a species, can be applied to prepare and deliver one or more selected genetic elements to a host organism that produces offspring and descendants that exhibit the preselected heritable trait. In some embodiments of the invention, the selected genetic elements introduced into a host organism are introduced into a reproductive cell in the host organism. In some aspects of the invention, a host cell or host organism that includes a composition of the invention may be referred to as a “modified” cell or “modified” organism, respectively.

A composition of the invention comprising one or more genetic elements selected to result in the inclusion of one or more preselected inheritable traits may be delivered to cell of a host organism, optionally when the cell is at a particular developmental stage. Non-limiting examples of cells and/or stages of cells to which a composition of the invention may be delivered or included are embryonic cells, germline cells, gametes, reproductive cells, cells that can give rise to a gamete, zygotes, pre-meiotic cells, post-meiotic cells, fully differentiated cells, and mature cells. Non-limiting examples of cells into which one or more selected genetic elements may be introduced in certain embodiments of the invention, are one or more of an isolated cell, a cell in a cell line, a cell in cell culture, a cell in tissue culture, a cell in an organ culture, and a cell that is within an organism. In certain embodiments of the invention, a cell is a zygote, a gamete, a cell that is able to give rise to a gamete, a germline cell, etc.

Compositions and methods of the invention may be delivered into and implemented in various types of cells and/or organisms. As used herein the term “subject” may be used to denote an organism that is prepared, introduced, detected, determined or otherwise subjected to an embodiment of a method of the invention. In some aspects of the invention, a cell or organism is a vertebrate or an invertebrate cell or organism. In certain aspects of the invention, a cell or organism is a eukaryotic or prokaryotic cell or organism. Non-limiting examples of organisms to which a method of the invention of the invention may be implemented are sexually reproducing organisms including but not limited to insects, fish, reptiles, amphibians, mammals, rodents, and birds. In some embodiments of the invention an organism is a mammal, including but not limited to cats, dogs, pigs, pigeons, starlings, fish (e.g., carp, trout, etc.), ferrets, weasels, stoats, possums, mongooses, mice, squirrels, rats, chipmunks, moles, voles, etc. In certain embodiments of the invention, an organism is not a human organism.

In some embodiments of methods of the intention, an organism is a rat, and optionally is a rat of the genus Rattus. In certain embodiments of methods of the invention, an organism is a mouse, and optionally is a mouse of the genus Mus or the genus Peromyscus. In some embodiments of the invention, an organism is a white-footed mouse (P. leucopus). In some embodiments of the invention, an organism is of the species Sus. In certain embodiments of the invention, an organism is of the order: Rodentia. An organisms that may be used in methods of the invention, for example though not intended to be limiting: as a host organism or as a mate for a host organism, include but are not limited to: a captive organism raised in captivity, a captive organism obtained from a wild population of organism of the species, a domesticated organism, a wild organism, a domesticated animal, an agricultural animal, a zoo animal, and a wild animal. Embodiments of methods and compositions of the invention can be used in endangered organisms in which the introduction of new genetics are beneficial to species survival. In some aspects of the invention, methods are provided that can be used in an endangered organism of a species in which one or more preselected genetic traits must be introduced to the species of organism to result in, or to aid in, survival of the species.

In some aspects of the invention, an organism is selected and a host organism prepared because the organism is the species as a population of organisms of interest to assess and study. A population of organisms may be of interest to study at least in part because of factors associated with the population, such as but not limited to: population size, geographic limitations of the population, geographic location of the population, environmental pressures on the population, status of the population as endangered, etc. In a non-limiting example, a population of organisms of interest may be a population of mice that is geographically isolated from other mouse populations. Embodiments of methods of the invention may comprise one or more of (1) releasing one or a plurality of host organisms into a population of the organism of interest and (2) releasing a plurality of offspring of one or a plurality of host organisms into a population of the organism of interest. A population of organisms of interest may be a local population, non-limiting examples of which include: a population in a geographically defined region, such as a forest, swamp, field, pond, island, building, etc. and a population in a politically defined region, such as a town, state, county, etc. In certain aspects of the invention, an organism species in which certain embodiments of methods of the invention may be implemented is an organism species that serves as a vector for disease affecting humans, animals, or plants. The term “vector” as used herein in reference to disease transfer, means an organism that carries and transmits an infectious pathogen into another living organism.

It will be understood that methods and compositions of the invention can be used alone or used in any combination of: before, simultaneously with, and after use of one or more alternative methods to assess, monitor, modify, and/or modulate a population of interest. In addition, methods of the invention, in some embodiments, may include introducing into a population of organism two or more preselected heritable phenotypic and/or genotypic traits. As a non-limiting example, one coat-color trait carried by a phenotypic allele, and a different coat-color trait carried by a different phenotypic allele, both coat colors distinguishable from a wild-type coat color and each other. In some embodiments of the invention, a first host organism is prepared using methods and compositions that result in offspring of the first host organism exhibiting a first inheritable coat-color trait. A second host organism is also prepared using methods and compositions and offspring of the second host organism exhibit a second inheritable coat-color heritable trait. The first and second host organism by be released into a population of interest and the coat color of organism in the population monitored as a measure of the gene flow and changes in the population. In certain embodiments of methods of the invention, a first host organism comprising one or more genetic elements that result in offspring of exhibiting a first preselected heritable trait and a second host organism comprising one or more genetic elements that result offspring that exhibit a second preselected heritable trait are released into a population of organism, or the offspring are released into a population of the organism. The releases of first and second host organisms and first and second resulting engineered offspring may be simultaneous, consecutive, repeated, alternating, or in any other manner deemed suitable to introduce two or more preselected heritable traits into the population of interest.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

### Introduction

The prospect of introducing engineered DNA into wild populations has been controversial due to the potential for unwanted ecological effects, in addition to objections over releasing large numbers of organisms viewed as pests. The Mice Against Ticks project, which aims to prevent tick-borne disease by heritably immunizing wild populations of white-footed mice on the islands of Nantucket and Martha's Vineyard, has found that these topics are of equivalent concern to most citizens. Similarly, discussions with Maori iwis and conservationists in Aotearoa New Zealand have identified that the prospect of introducing large numbers of additional rodents—even animals engineered to suppress and humanely remove the invasive population—may be viewed as problematic. It is also expected that release of captivity-bred organisms into wild environments would result in suboptimal survival and mating competence, and thus using such organisms would require unacceptably large numbers of the organism to be released in order to achieve the desired effects of introducing engineered DNA into a population. Drawbacks as describe above can be overcome using methods of the invention disclosed herein.

### Methods and Results

Studies are performed that include preparing an impregnated captive female organism of a species (also referred to as a “host organism”). One or more genetic elements have been introduced into the host organism and have been selected so that offspring of the host organism will exhibit a preselected heritable trait that can be detected and used to distinguish an organism that includes the preselected heritable trait from an organism that does not exhibit the preselected heritable trait. The exhibition of the one or more preselected heritable traits in an offspring of a host organism results from the one or more genetic elements introduced into the host organism. Because of the introduced genetic elements, offspring of the host organism have genetics comprising a gene and/or gene allele, the inclusion of which result in the offspring exhibiting the preselected heritable trait.

Offspring of host organisms prepared as described have different genetics than the genetics of the host organism. In some studies, the difference in the genetics of the host organism and its offspring is the presence of a gene or gene allele in the offspring that is not present in the genome of the host organism. Some experiments are performed in which one or more genetic elements are introduced into a host organism using genetic engineering methods. Certain experiments are performed in which one or more genetic elements are introduced into a host organism via mating with a male organism of the species. In some studies, the one or more genetic elements are introduction into a reproductive cell of the host organism.

Some experiments are performed in which one or more impregnated captive female organisms have genetics of a wild-type organism of the species. Offspring of the impregnated captive female organisms with wild-type genetics, have genetics that are wild-type with the exception of one or more genes or gene alleles that are present in the offspring because of the genetic elements introduced in to the host organism. One or more components of the introduced genetic elements are selected for inclusion because their presence in the host organism results in offspring of the host organism that that exhibit one or more preselected heritable traits.

Studies are performed in which one or more genetic elements that are introduced into the impregnated captive female organism are selected in part because their inclusion in the host organism results in offspring that exhibit one or more preselected heritable traits. The presence of the genetic elements in the host organism results in inclusion of one or more genes or gene alleles in offspring of the host organism. Offspring organisms are identified as having the same genetics of the host organism, but also include the one or more gene or gene alleles that result in the offspring exhibiting the one or more preselected heritable traits.

In the studies, the genetic elements are introduced into a prepared host organism and the host organism is introduced into a population of interest. The host organism give birth to the gestating offspring after release into the population of interest. The offspring and descendants of the impregnated captive female organism, exhibit the preselected heritable trait(s). Inclusion of the one or more genetic elements in the prepared impregnated female organism results in genetically engineered offspring and descendants of the prepared impregnated captive female organism. In some studies, it is identified that the introduction of the one or more genetic elements in the host organism results in her producing engineered offspring in which the genome of each engineered offspring and/or descendant is a the same as the host organism, except it includes the one or more genes or alleles.

In some experiments, a female organism that has the same genetics as an organism of a population of interest is obtained and prepared as the host organism. Results of these studies demonstrate that the engineered offspring produced by the host organism have genetics essentially identical to the genetics of the organism of the initial population of interest, except it includes the one or more genes or alleles.

In additional studies, a preselected heritable trait is introduced into a population of interest and later the trait is detected in organisms in the population. The detection of the preselected heritable trait in an organism in the population identifies the organism as an engineered organism. The determination that an organism in the population does not exhibit the preselected heritable trait identifies that organism as a non-engineered organism.

A plurality of prepared impregnated captive wild-type female organisms is released into a population of the same species of organisms. Offspring and descendants of the released impregnated female organism are genetically engineered organisms and have primarily wild-type genomes but their genomes also include genes or gene alleles responsible for a preselected heritable trait. The genes and/or gene alleles in the offspring result from the introduction of the genetic element(s) in the impregnated captive female organism. The prepared host organism are released into a population of interest and the genetically engineered organism exhibit robust survival and mating competence. Experiments also include use of genetic engineering methods to introduce genetically engineered organisms of a species into a population of interest wherein the genetically engineered organism are distinguishable from non-engineered organism in the population.

### Example 2

Studies are performed in M. musculus in which visible dominant (blaze) and recessive (Oca2^{p}, Tyr^{c-ch}) coat color alleles are introduce and tracked. These preselected heritable coat color alleles are introduced into one or more wild populations inhabiting urban buildings in a selected community. Continuous live trapping in all buildings is performed and generates a comprehensive dataset of recent gene flow patterns to supplement sequencing-based efforts. The studies compare two methods: (1) capturing wild females, mating them to captive males carrying these alleles, and (2) releasing the pregnant females at the sites of capture, or allowing them to give birth in captivity and releasing the grown offspring.

At several times after the introduction, organisms in the population that exhibit the preselected heritable trait (also referred to as engineered organisms) are detected. Numbers, geographic distribution, and other characteristics of engineered organism in the population are determined and assessed over time. Data on gene flow and dispersal of the engineered organism in the population is collected. Changes in characteristics such as the relative number of engineered organisms compared to the number of non-engineered organisms in the population, the geographic location of engineered organisms, and other characteristics are determined.

### Example 3

Studies are performed in which preselected heritable alleles from a first population of organisms of a species are introduced into a different, geographically separated second population of organisms of the same species. An additional study is also performed in which one or more preselected heritable alleles from the second population of organisms of a species are introduced into the first population of organisms. A variety of SNPs are detected and tracked using gene sequencing. In this study, results to indicate differences in gene flow, stability, etc. in the first versus the second populations.

Organisms used in the studies are P. leucopus. One or more preselected heritable trait is introduced into a wild population of P. leucopus, using methods as described in Example 1. In some instances, the preselected heritable trait include one or both of resistance to tick-borne disease and resistance to ticks. The genetic elements that are delivered into an organism result in offspring of the organism that exhibit one or both of the preselected phenotypic traits of resistance to tick-borne diseases and/or resistant to ticks. At several times after the introduction, organisms in the population that exhibit the preselected heritable trait (also referred to as engineered organisms) are detected. Numbers, geographic distribution, and other characteristics of engineered organism in the population are determined and assessed over time. Data on gene flow and dispersal of the engineered organism in the population is collected. Changes in characteristics such as the relative number of engineered organisms compared to the number of non-engineered organisms in the population, the geographic location of engineered organisms, and other characteristics are determined. A result of the studies indicate that the genetic elements delivered into an organism result in offspring of the organism that exhibit one or both of the preselected phenotypic traits of resistance to tick-borne diseases and/or resistant to ticks.

### Results of Examples 2 and 3

The results of the studies empirically illuminate gene flow patterns in the two wild rodents and identifies efficient methods of introducing novel genes.

### Example 4

Studies are performed in M. musculus and/or R. norvegicus and/or R. rattus in which an engineered Y chromosome that results in the inviability or infertility of daughter organisms is introduced into a population. Due to this phenotype, only the sons of released female organisms are fertile and capable of passing on the engineered Y chromosome. The introduction of this preselected engineered Y chromosome into a wild population inhabiting urban buildings in a selected community is anticipated to result in a durable reduction of the number of fertile females and the fecundity of the local wild population. Continuous live trapping in all buildings is performed and generates a comprehensive dataset of recent gene flow patterns by carriers of the engineered Y chromosome. The study is supplemented by catching carrier males when young and attaching tracking devices to monitor their movements in areas with abundant resources but few fertile females. Additional copies of the engineered Y chromosome are introduced into the population and the abundance assessed by trapping.

### Example 5

### Preparing Engineered Male-Linked Daughterless Mice

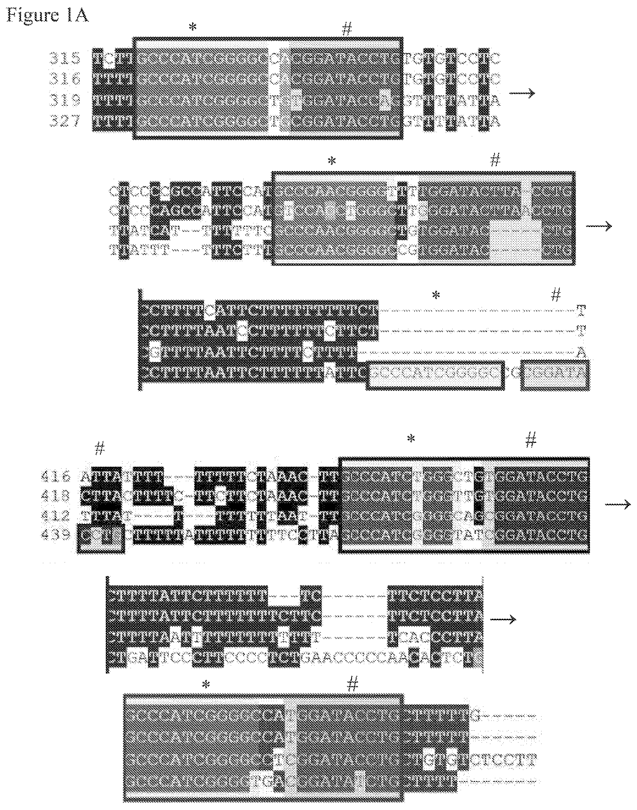

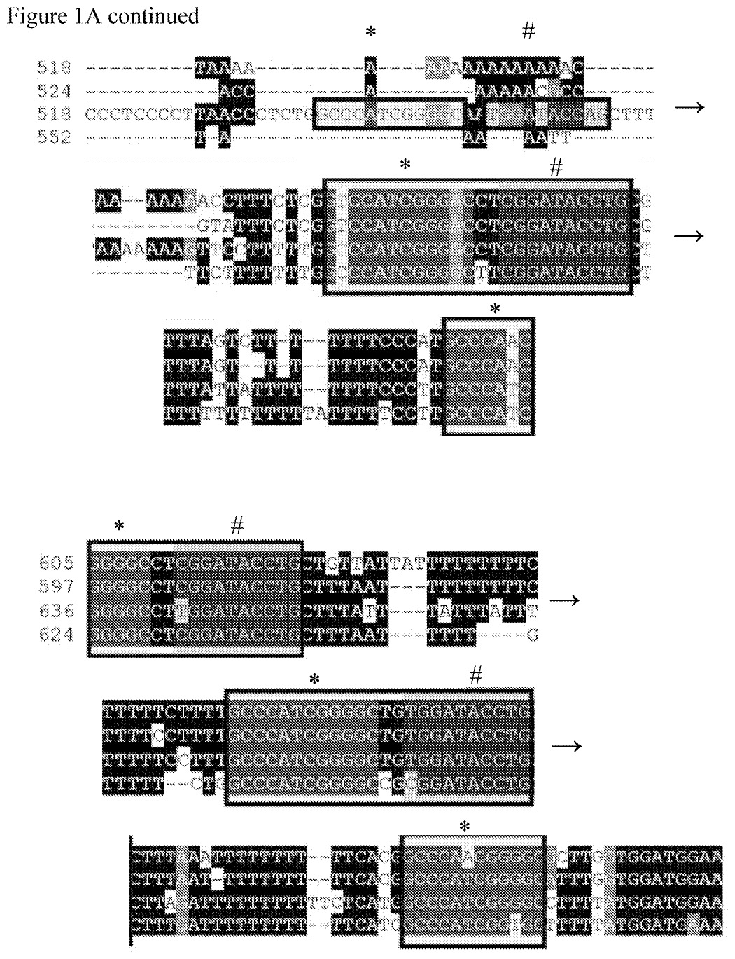

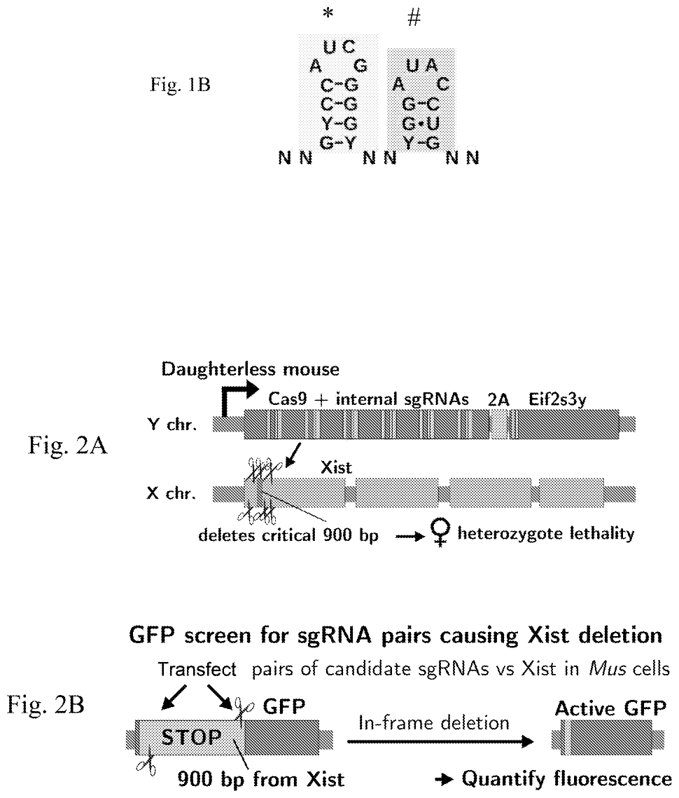

This approach involves encoding an RNA-guided nuclease and guide RNA system on the Y chromosome to knock out a critical 900 base-pair region of the female-specific noncoding RNA Xist (FIGS. 1-2). Loss of Xist function has no known effects in male mice, nor in daughters who inherit a dysfunctional copy from their mother. However, loss of function mutations in the paternal copy of Xist are selectively lethal in daughters at embryonic day 8.5 due to failed X-inactivation in the trophoblast [Marahrens, Y., et al., (1997) Genes & Development 11 (2): 156-66; Wutz, A. et al., (2002), Nature Genetics, 30(2):167-174; Hoki et al. (2009), Development, 136(1):139-146].

This method involves the deletion of a small segment of Xist exon 1 that encodes critical repeated stem-loop motifs, a minimal number of which are strictly required for X-inactivation in mice (FIG. 1) [Wutz, A., et al., (2002) Nature Genetics 30 (2): 167-174; Hoki et al. (2009), Development, 136(1):139-146]. Expressing an RNA-guided nuclease and multiple guide RNAs targeting sequences flanking or distributed throughout this region therefore yields a targeted deletion and loss of Xist function. Because Xist is highly conserved across mammals (FIG. 1), this deletion method can be similarly effective across rodents [Nesterova, T. B., et al., (2001) Genome Research 11 (5): 833-49] and other mammalian invasive species and pests [Loda, A., & Heard, E. (2019) PLoS Genetics 15 (9): e1008333].

There are many possible molecular implementations of a Y-linked system capable of disrupting Xist functionality on the X chromosome. An embodiment of a system of the invention comprises encoding an RNA-guided nuclease and multiple guide RNAs targeting sequences flanking this region, such that nuclease activity occurs in male germline cells prior to spermiogenesis. Cutting multiple sites in flanking regions and within the relevant region leads to targeted deletions when repaired by non-homologous end-joining. Restricting activity to the germline cells minimizes potential fitness costs of nuclease expression. The nuclease is encoded as an N-terminal 2A-peptide fusion [Trichas, G., et al., (2008) BMC Biology 6 (September): 40; Liu, Z., et al., (2017) Scientific Reports 7 (1): 2193] to the Y chromosome-encoded protein Eif2s3y expressed in the male germline. Eif2s3y is expressed throughout male germline development, allowing ample time for the nuclease to disrupt Xist [Mazeyrat et al. (2001), Nature Genetics, 29(1):49-53]. A 2A peptide fusion to a Y-chromosomal gene is desirable because it provides reliable expression in the male germline [Mazeyrat et al. (2001), Nature Genetics, 29(1):49-53; Armoskus et al. (2014), Brain Research, 1562:23-38; Huby, R. D. J. et al., (2014), PloS one, 9(12):e115792; Li et al. (2016), Oncotarget, 7(10):11321-11331]. To compensate for the reduced expression of the Y-chromosomal gene resulting from inefficient translational re-initiation after the 2A peptide, the Kozak sequence controlling translational initiation is strengthened to the consensus sequence, and introns are inserted into the nuclease gene to enhance transcription. Any nonsense mutation in the sequence encoding the nuclease will be selected against due to the accompany loss of function of Eif2s3y, which results in male sterility.

Other Y-chromosomal genes expressed in the male germline may be used in place of Eif2s3y; for example, Zfy2 provides a narrow burst of expression during late meiosis. Alternatively, the nuclease may be encoded with its own expression signals away from existing endogenous Y chromosomal genes, although this risks poor activity and loss-of-function mutations in the nuclease. Generally, any method of expression that results in sufficient expression of the nuclease to achieve Xist deletion in the male germline is adequate for the purpose; restricting expression is primarily useful to minimize potential fitness costs resulting from expression of the nuclease in all cells rather than only germline cells.

To produce functional guide RNAs within the same cassette that encodes the nuclease, polymerase III promoters expressing guide RNAs targeting Xist are embedded within introns that are inserted into the coding region of the nuclease (FIG. 2A). This approach is analogous to natural arrangements in which tRNAs are expressed from within the introns of functional protein-coding genes. While the guide RNAs could be encoded elsewhere on the Y chromosome, this arrangement permits a daughterless male to be generated with a single insertion step. Crucially, paternal deposition of nuclease in sperm is not required for the success of this design, nor is it harmful should this occur because Xist disruption has no effect in males. This approach does not involve sperm-killing, meaning that it is not be affected by polyandry [Manser, A., et al., (2019) Proceedings. Biological Sciences/The Royal Society 286 (1909): 20190852]. All sperm from daughterless males are equally fertile and competitive, but embryos arising from sperm that carry a female-determining X chromosome are eliminated at embryonic day 8.5, before the development of the nervous system and the onset of pain perception [Marahrens, Y., et al., (1997) Genes & Development 11 (2): 156-66].

Engineered male organisms are prepared in which an RNA-guided nuclease and guide RNA system that knocks out the critical 900 base-pair region of the female-specific noncoding RNA Xist are encoded on the Y chromosome. The daughterless-male trait is delivered into a target population of the organisms by one or both of: (1) releasing the engineered male into a target population and (2) delivering genetic material from the engineered male into one or a plurality of captive females of the organism in a manner that results in impregnation of the female(s), and releasing the impregnated female(s) into a target population of the organisms.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed:

1. A method of changing a genetic characteristic of a target population of organisms of a species, comprising:
(a) preselecting a heritable trait for introduction into a target population of organisms; and
(b) introducing by birth into the target population an engineered organism comprising the preselected heritable trait, wherein the introduction changes a genetic characteristic of the target population.

2. The method of claim 1, wherein a means of introducing the preselected heritable trait by birth comprises:
(i) delivering into a reproductive cell of a captive female of the organism a gene or gene allele that when expressed in an descendant of the captive female organism results in an engineered organism that exhibits the preselected heritable trait;
(ii) impregnating the captive female of the organism; and
(iii) releasing the impregnated captive female of the organism into the target population of organisms.

3. The method of claim 2, wherein the means of the delivery in (a) impregnates the captive female.

4-7. (canceled)

8. The method of claim 1, wherein the engineered organism is a male engineered organism.

9-14. (canceled)

15. The method of claim 1, wherein the organism is a mammal, and optionally is of the genus Rattus, Mus, Sus, Felis, or Canis.

16-20. (canceled)

21. The method of claim 15, wherein the preselected heritable trait comprises a coat color of the organism, and results from expression of a coat-color allele.

22-23. (canceled)

24. The method of claim 1, wherein the preselected heritable trait is a daughterless-male heritable trait.

25. The method of claim 24, wherein the daughterless-male heritable trait is generated by engineering a Y chromosome that when present in a female of the organism disrupts an activity of an X chromosome non-coding RNA gene.

26. The method of claim 24, wherein the daughterless-male heritable trait comprises disruption of an activity of an X chromosome non-coding RNA gene in female descendants of an engineered male organism, wherein the disruption of the activity of the X chromosome non-coding RNA gene in the female descendants is embryonically lethal to the female descendants.

27. The method of claim 26, wherein a means of producing the descendant organisms comprising the heritable trait comprises impregnating a female organism of the species with genetic material of the engineered male.

28. (canceled)

29. The method of claim 25, wherein a means for the disrupting of an activity of the X chromosome non-coding RNA gene in female descendants of the engineered male organism comprises encoding on the engineered male organism's Y chromosome a nuclease capable of disrupting the activity of the X chromosome non-coding RNA gene.

30-51. (canceled)

52. The method of claim 1, wherein the preselected heritable trait is a sterile-daughter heritable trait.

53. The method of claim 52, wherein the sterile-daughter heritable trait is generated by engineering a Y chromosome that disrupts an activity of an X chromosome gene required for the fertility of female offspring.

54. The method of claim 52, wherein the sterile-daughter heritable trait is generated by engineering a Y chromosome that produces a nuclease carried in sperm of the engineered male organism and when present in a zygote, the nuclease cuts one or more female-specific fertility genes.

55-59. (canceled)

60. The method of claim 2, wherein the delivering of the gene or gene allele into the reproductive cell of the captive female organism comprises an in vitro fertilization (IVF) method, an artificial insemination method, or mating of the captive female organism with a male organism of the species.

61-62. (canceled)

63. The method of claim 1, further comprising detecting the preselected heritable trait in an organism in the target population following the introduction by birth of the preselected heritable trait into the target population.

64-72. (canceled)

73. The method of claim 2, further comprising, capturing one or more female organisms from the target population, at a time subsequent to the release of the impregnated captive female, wherein the capturing is aided in part by a determined spatial distribution of engineered organisms introduced by birth into the target population.

74-75. (canceled)

76. A method of altering a genetic characteristic of a target population of organisms of a species, comprising:
a) obtaining one or a plurality of captive female organisms of a species, wherein the species is a non-human species;
b) introducing a genetic element from one or more male organisms of the species into the one or the plurality of the captive female organisms, wherein each of the one or more male organisms exhibits one or more preselected heritable traits; wherein the introducing results in impregnation of at least one of the captive female organisms, and a descendant of the impregnated captive female organism exhibits one or more of the preselected heritable traits;
c) identifying one or more of the captive female organisms impregnated in (b); and
d) releasing one or more of the identified impregnated captive female organisms into a target population of organisms of the species, wherein the presence of one or more descendants of the impregnated captive female organisms alters a genetic characteristic of the target population.

77. The method of claim 76, wherein the preselected heritable trait comprises one or more of a preselected heritable phenotypic trait and a preselected heritable genotypic trait.

78-80. (canceled)

81. The method of claim 76, wherein the organism is a rodent.

82-120. (canceled)
